(19) United States
(12) Patent Application Publication
(10) Pub. No.: US 2023/0272096 A1
(43) Pub. Date: Aug. 31, 2023

(54) Treatment Of Lung Disease Based Upon Stratification Of Polygenic Risk Score For Interleukin 33 (IL-33)

(71) Applicant: Regeneron Pharmaceuticals, Inc., Tarrytown, NY (US)

(72) Inventors: Charles Paulding, Tarrytown, NY (US); Shan Chen, Tarrytown, NY (US)

(21) Appl. No.: 18/054,374

(22) Filed: Nov. 10, 2022

Related U.S. Application Data
(60) Provisional application No. 63/278,266, filed on Nov. 11, 2021.
(60) Provisional application No. 63/351,848, filed on Jun. 14, 2022.

(51) Int. Cl. C07K 16/28; C07K 16/24; A61P 11/06; G01N 33/68

(52) U.S. Cl. CPC C07K 16/2866; C07K 16/244; A61P 11/06; G01N 33/6869; G01N 2800/52; G01N 2333/54

(57) ABSTRACT

The present disclosure provides methods of treating lung disease subjects that are likely to respond to an IL-33 antagonist, an interleukin-4 receptor alpha antagonist, or an IL-13 receptor antagonist, based on the subject's IL-33 Asthma polygenic risk score.

### REFERENCE TO SEQUENCE LISTING

This application includes a Sequence Listing filed electronically as an XML file named 381203559SEQ, created on Nov. 9, 2022, with a size of 14 kilobytes. The Sequence Listing is incorporated herein by reference.

### FIELD

The present disclosure relates to the field of therapeutic treatments of lung diseases. More specifically, the disclosure relates to methods of increasing the efficacy of interleukin-33 (IL-33) antagonist, interleukin-4 receptor alpha antagonist, and/or interleukin-13 receptor antagonist therapies in lung disease subjects by identification of subjects that are likely to respond to an IL-33 antagonist, an interleukin-4 receptor alpha antagonist, and/or an interleukin-13 receptor antagonist.

### BACKGROUND

Asthma is an inflammatory disease of the airways of the lungs. The condition is characterized by variable and recurring symptoms, reversible airflow obstruction, and bronchospasms that are easily triggered. The diagnosis of asthma can involve spirometry lung function testing, including determination of the forced expiratory volume in one second (FEV1), the peak expiratory flow rate, and assessment of the annualized exacerbation rate. Treatments of asthma include administration of medications that are fast acting or effective in the longer term. Salbutamol and albuterol are the mainstays of fast-acting medications whereas inhaled corticosteroids (ICSs) were for many years the cornerstone of long-term therapies. More recently, antibodies such as mepolizumab, dupilumab, and omalizumab have been used in connection with specific types of asthma. However, it is difficult to predict whether a particular subject will respond to a particular antibody therapy. It is also difficult to predict the annualized exacerbation rate or the loss of asthma control (LOAC) prospectively.

Chronic obstructive pulmonary disease (COPD) is characterized in part by air-flow restriction, emphysema, and chronic bronchitis. COPD frequently worsens with everyday activity making routine tasks difficult. While tobacco smoke is a major risk factor, other factors include pollution, genetics, and exposure to workplace dusts and chemicals. Diagnosis of COPD frequently comprises spirometry including determination of a patient's FEV1 value. Current treatments include smoking cessation, short-acting bronchodilators, phosphodiesterase-4 inhibitors, corticosteroids, and, in serious cases, antibiotics. However, it is difficult to predict whether a patient will respond to a particular treatment.

### SUMMARY

The present disclosure provides methods of treating a subject having a lung disease, such as asthma and COPD, or at risk of developing a lung disease, such as asthma and COPD, the method comprising: administering an IL-33 antagonist to the subject when the subject's IL-33 asthma polygenic risk score (IL-33 Asthma PRS) is greater than or equal to a threshold IL-33 Asthma PRS, or administering an interleukin-4 receptor alpha antagonist and/or an interleukin-13 receptor antagonist to the subject when the subject's IL-33 Asthma PRS is less than a threshold IL-33 Asthma PRS, wherein the IL-33 Asthma PRS comprises a weighted aggregate of a plurality of genetic variants near or in the IL-33 and IL1RL1 genes associated with asthma.

The present disclosure also provides methods of treating a subject having a lung disease, such as asthma and COPD, or at risk of developing a lung disease, such as asthma and COPD, the method comprising: administering an interleukin-4 receptor alpha antagonist and/or an interleukin-13 receptor antagonist to the subject when the subject's IL-33 Asthma PRS is greater than or equal to a threshold IL-33 Asthma PRS and administering a standard amount or greater of a composition for treating asthma exacerbation, wherein the IL-33 Asthma PRS comprises a weighted aggregate of a plurality of genetic variants near or in the IL-33 and IL1RL1 genes associated with asthma.

The present disclosure also provides methods of determining whether a subject should be administered an interleukin-4 receptor alpha antagonist, an interleukin-13 receptor antagonist, and/or an IL-33 antagonist for treatment of a lung disease, such as asthma and COPD, the method comprising: determining or having determined the subject's IL-33 asthma polygenic risk score (IL-33 Asthma PRS), wherein the IL-33 Asthma PRS comprises a weighted aggregate of a plurality of genetic variants near or in the IL-33 and IL1RL1 genes associated with asthma; and determining or having determined whether the subject's IL-33 Asthma PRS is greater than or equal to a threshold IL-33 Asthma PRS; and when the subject's IL-33 Asthma PRS is greater than or equal to the threshold IL-33 Asthma PRS, the subject should be administered the IL-33 antagonist; or when the subject's IL-33 Asthma PRS is less than the threshold IL-33 Asthma PRS, the subject should be administered the interleukin-4 receptor alpha antagonist and/or the interleukin-13 receptor antagonist.

The present disclosure also provides methods of determining whether a subject having asthma should be administered a standard amount or greater of a composition for treating asthma exacerbation, the method comprising: determining or having determined the subject's IL-33 asthma polygenic risk score (IL-33 Asthma PRS), wherein the IL-33 Asthma PRS comprises a weighted aggregate of a plurality of genetic variants near or in the IL-33 and IL1RL1 genes associated with asthma; and when the subject's IL-33 Asthma PRS is greater than or equal to a threshold IL-33 Asthma PRS, the subject should be administered a standard amount or greater of a composition for treating asthma exacerbation.

### BRIEF DESCRIPTION OF THE DRAWINGS

The accompanying figures, which are incorporated in and constitute a part of this specification, illustrate several features of the present disclosure.

The patent or application file contains at least one drawing executed in color. Copies of this patent or patent application publication with color drawing(s) will be provided by the Office upon request and payment of the necessary fee.

FIG. 1 (Panels A and B) shows significant genetic association with annualized asthma exacerbation rate. Specifically, IL33 PRS quartile is associated with the subject patient's response to dupilumab treatment efficacy.

FIG. 2 (Panels A and B) shows measuring a co-primary endpoint of FEV1 change. The genetic effect is not significant for those on the same treatment group.

FIG. 3 shows that when comparing dupilumab treatment and placebo arms, the higher IL33 PRS quartile subjects (75 and 100 groups) are significantly associated with less response efficacy to dupilumab.

FIG. 4 (Panels A and B) shows that IL-33 Asthma PRS determined using the LDpred method was not associated with a change of FEV1 at week 12 in dupilumab-treated admixed American patients.

FIG. 5 shows that IL-33 Asthma PRS determined using the LDpred method showed a trend for decreased loss of asthma control (LOAC) risk in itepekimab-monotreated European subjects.

FIG. 6 shows that IL-33 Asthma PRS showed a trend for decreased LOAC risk in itepekimab-monotreated European and admixed American subjects.

FIG. 7 (Panels A and B) shows that IL-33 Asthma PRS determined using the LDpred method was significantly associated with asthma in GHS_GSA European subjects.

FIG. 8 (Panels A and B) shows that IL-33 Asthma PRS determined using the LDpred method was significantly associated with eosinophil count in GHS_GSA European subjects.

FIG. 9 shows that 11 out of 543 variants in an IL-33 Asthma PRS determined with LDpred training were disease- or trait-associated variants in the HGMD.

FIG. 10 (Panels A and B) shows that IL-33 asthma polygenic risk score for admixed American subjects (IL-33 AMR Asthma PRS) determined using the LDpred method was significantly associated with asthma risk in Mexico City admixed American subjects.

FIG. 11 shows that 6 out of 205 variants in an IL-33 AMR Asthma PRS determined with LDpred training were associated with diseases or traits in the HGMD.

FIG. 12 (Panels A and B) describes IL-33 Asthma PRSs determined with LDpred training in a trial of itepekimab-treated European and admixed American combined subjects.

FIG. 13 shows that IL-33 Asthma PRS trends for decreased LOAC risk in itepekimab-monotreated European and admixed American subjects.

FIG. 14 shows that IL-33 Asthma PRS is associated with an increased baseline basophil count for European subjects in trials designated DR112544 and EFC13579.

FIG. 15 shows that IL-33 Asthma PRS was significantly associated with increased asthma exacerbation in dupilumab-treated European subjects in trials designated DR112544 and EFC13579.

FIG. 16 (Panels A and B) shows a combined IL-33 Asthma PRS determined with LDpred training for European and admixed American subjects in trials designated DR112544 and EFC13579.

FIG. 17 (Panels A and B) shows that patients having IL-33 Asthma PRS score in the top 25% responded to dupilumab with less efficacy, compared to patients having lower IL-33 Asthma PRS scores and treated with dupilumab; patients are from European and admixed American combined subjects in trials designated DR112544 and EFC13579.

FIG. 18 (Panels A and B) shows that patients having IL-33 Asthma PRS score in the bottom 25% in the dupilumab arm have the most increased value of FEV1 compared to placebo arm; (0.3-0.14) has the most difference compared to other quartile groups.

FIG. 19 (Panels A and B) shows a relationship between IL-33 Asthma PRS and a change of FVC at week 12 in European and admixed American subjects.

FIG. 20 shows the absence of a relationship between IL-33 Asthma PRS and the proportion of GHS_GSA European subjects with asthma exacerbation (Panel A), and the absence of a relationship between IL-33 Asthma PRS and the proportion of subjects taking asthma medications (Panel B).

FIG. 21 shows the relationship between IL-33 Asthma PRS and the proportion of GHS_GSA European patients with COPD.

### DESCRIPTION OF EMBODIMENTS

Genetic factors can play an important role in a risk of developing a disease and potentially influence how individuals respond to drug treatment. Polygenic risk scores (PRSs) combine information from a large number of genetic variants derived from disease association studies to create a single composite quantitative measure for each individual which reflects their genetically-derived disease risk. An individual with a larger number of risk alleles for a particular disease will have a higher PRS than an individual with fewer alleles for the same particular disease. Risk can be evaluated at several thresholds, such as percentiles, standard deviation units of the population distribution, or absolute values. The present disclosure relates generally to the unexpected finding that stratification of subjects by IL-33 Asthma PRS is useful in the identification of subjects likely to respond to an IL-33 antagonist, an interleukin-4 receptor alpha antagonist, and/or an interleukin-13 receptor antagonist in the treatment of a lung disease, such as asthma and COPD.

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

The present disclosure relates generally to methods and compositions for treating a subject having a lung disease, such as asthma and COPD, or at risk of developing a lung disease, such as asthma and COPD. In some embodiments, the lung disease comprises asthma. In some embodiments, the lung disease comprises COPD.

Without being limited by any particular theory, it is believed that the IL-33 Asthma PRS calculated according to the methods presented herein allow for identification of subjects likely to respond to an IL-33 antagonist, an interleukin-4 receptor alpha antagonist, and/or an interleukin-13 receptor antagonist. Furthermore, surprisingly and unexpectedly, the IL-33 Asthma PRS is also predictive of a subject's response to an IL-33 antagonist, an interleukin-4 receptor alpha antagonist, and/or an interleukin-13 receptor antagonist.

In some embodiments, a subject who is treatable by the methods of the present disclosure has had asthma within the past 1, 2, 3, 4, 5, 6, 7, 8, 9, 10, 11, 12, 13, 14, 15, 16, 17, or 18 months. The subjects who are treatable by the methods of the present disclosure include subjects that have been hospitalized with asthma-related symptoms and subjects that are currently hospitalized.

In some embodiments, a subject who is treatable by the methods of the present disclosure has had COPD within the past 1, 2, 3, 4, 5, 6, 7, 8, 9, 10, 11, 12, 13, 14, 15, 16, 17, or 18 months. The subjects who are treatable by the methods of the present disclosure include subjects that have been hospitalized with COPD-related symptoms and subjects that are currently hospitalized.

In some embodiments, the subject may be selected on the basis of an IL-33 Asthma PRS, wherein the IL-33 Asthma PRS comprises an aggregate or weighted aggregate of a plurality of genetic variants associated with asthma or COPD, and is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 2,000, at least about 3,000, at least about 4,000, at least about 5,000, at least about 6,000, at least about 7,000, at least about 8,000, at least about 9,000, or at least about 10,000 genetic variants. If the subject has an IL-33 Asthma PRS greater than or equal to a threshold IL-33 Asthma PRS, the subject should be administered an IL-33 antagonist. Alternatively, if the subject has an IL-33 Asthma PRS less than a threshold IL-33 Asthma PRS, the subject should be administered an interleukin-4 receptor alpha antagonist and/or an interleukin-13 receptor antagonist. In some embodiments, the aggregate of a plurality of genetic variants associated with asthma is a weighted aggregate of a plurality of genetic variants associated with asthma. In some embodiments, the genetic variants are chosen from any one or more of the variants listed in Table 1 (variant id is according to GRCh38/hg38 human genome assembly coordinates).

TABLE 1

| variant_id | effective_allele | weight |
|---|---|---|
| 9:6128897:T:C | C | 6.39E−05 |
| 9:6134510:G:A | G | 2.75E−06 |
| 9:6233221:A:C | C | 3.44E−05 |
| 9:6213705:A:G | A | 5.84E−05 |
| 9:6247117:A:G | G | 2.41E−05 |
| 9:6254208:C:T | T | 1.91E−05 |
| 9:6256529:A:C | C | 1.75E−05 |
| 9:6185295:C:T | T | 0.00011693 |
| 9:6160578:C:T | T | 4.77E−05 |
| 9:6184165:T:C | T | 5.04E−05 |
| 9:6270359:A:G | A | 5.54E−06 |
| 9:6198384:A:G | G | 5.68E−05 |
| 9:6198894:C:T | T | 9.68E−06 |
| 9:6277389:T:C | C | 1.45E−05 |
| 9:6132625:G:A | A | 2.77E−06 |
| 9:6157329:C:T | T | 2.42E−05 |
| 9:6290673:T:C | T | 8.29E−06 |
| 9:6145491:T:C | C | 1.44E−05 |
| 9:6247430:C:T | T | 4.52E−05 |
| 9:6130822:C:T | T | 3.60E−05 |
| 9:6215336:T:C | T | 7.38E−06 |
| 9:6142948:C:A | A | 5.84E−05 |
| 9:6262338:G:A | A | 2.72E−05 |
| 9:6256292:G:A | G | 1.44E−05 |
| 9:6162489:C:T | C | 7.72E−06 |
| 9:6264432:T:C | C | 1.87E−05 |
| 9:6162881:T:C | T | 8.29E−06 |
| 9:6151320:C:A | A | 2.42E−05 |
| 9:6160783:A:G | G | 1.07E−05 |
| 9:6161686:C:T | T | 5.01E−05 |
| 9:6226289:A:G | A | 9.43E−06 |
| 9:6275119:G:A | A | 1.60E−05 |
| 9:6155865:C:T | T | 4.85E−05 |
| 9:6174316:T:C | T | 8.35E−05 |
| 9:6142157:A:C | C | 2.67E−05 |
| 9:6166653:G:A | A | 4.35E−05 |
| 9:6187862:T:C | T | 5.49E−05 |
| 9:6273645:C:T | C | 4.34E−06 |
| 9:6166919:T:C | T | 3.10E−05 |
| 9:6170914:T:C | T | 5.63E−07 |
| 9:6201364:G:A | G | 5.59E−05 |
| 9:6268569:T:C | C | 1.97E−05 |
| 9:6208855:A:G | G | 7.47E−05 |
| 9:6193022:T:C | T | 5.61E−05 |
| 9:6134999:C:T | T | 3.47E−05 |
| 9:6154139:T:G | T | 4.66E−05 |
| 9:6237263:G:A | A | 2.26E−05 |
| 9:6245971:G:A | A | 8.16E−06 |
| 9:6291242:T:C | T | 6.80E−06 |
| 9:6237360:G:A | G | 3.74E−05 |
| 9:6279987:T:C | T | 5.69E−06 |
| 9:6187242:A:G | A | 3.82E−05 |
| 9:6192796:T:C | T | 0.00013064 |
| 9:6153485:T:C | C | 4.80E−05 |
| 9:6202701:A:C | A | 3.99E−05 |
| 9:6199285:G:A | G | 4.42E−05 |
| 9:6187324:T:G | T | 9.84E−06 |
| 9:6269689:T:C | C | 1.58E−05 |
| 9:6158778:T:C | C | 2.56E−05 |
| 9:6170847:C:T | C | 0.00011106 |
| 9:6219176:G:T | T | 0.00012036 |
| 9:6234131:G:A | A | 1.26E−05 |
| 9:6261153:A:G | G | 1.90E−05 |
| 9:6240684:C:T | C | 1.05E−05 |
| 9:6225659:G:A | A | 5.05E−05 |
| 9:6236830:C:T | T | 7.09E−05 |
| 9:6197408:A:G | A | 3.59E−05 |
| 9:6161095:T:C | T | 2.65E−05 |
| 9:6264410:A:G | G | 3.03E−05 |
| 9:6157433:C:T | T | 2.28E−05 |
| 9:6266037:T:C | C | 3.09E−05 |
| 9:6213387:G:A | G | 0.00012989 |
| 9:6226592:C:T | T | 5.96E−07 |
| 9:6193455:C:A | C | 0.00012888 |
| 9:6258932:T:C | C | 1.87E−05 |
| 9:6194831:C:A | C | 8.81E−05 |
| 9:6271238:A:G | A | 5.94E−06 |
| 9:6176534:T:C | C | 3.73E−05 |
| 9:6268893:G:A | G | 4.13E−06 |
| 9:6260411:A:G | A | 3.59E−06 |
| 9:6145022:C:T | T | 1.32E−05 |
| 9:6135935:A:C | C | 2.46E−05 |
| 9:6163764:G:A | A | 6.07E−05 |
| 9:6255789:A:C | A | 1.49E−05 |
| 9:6171296:C:T | C | 1.14E−05 |
| 9:6149006:A:G | A | 1.09E−05 |
| 9:6227418:G:A | A | 4.56E−05 |
| 9:6227111:G:A | A | 3.97E−05 |
| 9:6238629:G:A | A | 2.79E−05 |
| 9:6222553:G:A | A | 2.92E−05 |
| 9:6280246:A:G | A | 7.83E−05 |
| 9:6134787:T:C | C | 7.30E−06 |
| 9:6268707:T:C | T | 9.07E−05 |
| 9:6155014:G:A | A | 2.34E−05 |
| 9:6240658:T:C | C | 1.20E−05 |
| 9:6243119:A:G | G | 7.61E−05 |
| 9:6208030:G:T | T | 0.00012809 |
| 9:6209697:A:G | A | 0.00013787 |
| 9:6289504:C:T | T | 1.56E−05 |
| 9:6161253:C:T | T | 2.55E−05 |
| 9:6231239:A:G | A | 3.41E−05 |
| 9:6202852:C:A | A | 8.25E−05 |
| 9:6240236:T:C | C | 7.60E−05 |
| 9:6288915:T:C | C | 1.56E−05 |
| 9:6219845:A:C | A | 3.43E−05 |
| 9:6227752:A:G | G | 1.10E−06 |
| 9:6214347:T:C | T | 4.03E−05 |
| 9:6196645:T:C | T | 8.89E−05 |
| 9:6185360:T:C | T | 3.79E−05 |
| 9:6247551:C:T | C | 2.06E−05 |
| 9:6159758:C:T | T | 5.40E−05 |
| 9:6144567:T:C | C | 1.37E−05 |
| 9:6144025:A:G | A | 7.38E−06 |
| 9:6154874:C:T | T | 6.98E−05 |
| 9:6234125:T:C | C | 3.45E−05 |
| 9:6243645:G:A | A | 2.61E−05 |
| 9:6201163:T:C | C | 0.00011843 |
| 9:6255152:A:G | G | 1.87E−05 |
| 9:6232242:C:T | T | 1.46E−05 |
| 9:6197377:A:G | G | 0.00012538 |
| 9:6283418:A:G | G | 1.27E−05 |
| 9:6235343:C:T | T | 1.26E−05 |
| 9:6159759:A:G | G | 4.71E−05 |
| 9:6213985:C:T | C | 8.17E−06 |
| 9:6188740:A:C | A | 8.91E−05 |
| 9:6257724:A:G | G | 3.00E−05 |
| 9:6170924:A:C | A | 4.24E−05 |
| 9:6224308:T:C | C | 3.21E−05 |
| 9:6191105:A:C | A | 5.51E−05 |
| 9:6187395:G:T | G | 3.67E−05 |
| 9:6267718:C:T | T | 3.05E−05 |
| 9:6264055:T:C | C | 3.00E−05 |
| 9:6276733:C:T | T | 1.52E−05 |
| 9:6257367:T:C | C | 2.97E−05 |
| 9:6282794:T:C | T | 1.05E−05 |
| 9:6216815:G:A | G | 1.63E−05 |
| 9:6230947:A:G | G | 2.60E−05 |
| 9:6281211:C:T | T | 1.27E−05 |
| 9:6211813:T:C | C | 0.00011837 |
| 9:6135411:C:A | A | 5.22E−06 |
| 9:6135847:C:T | T | 3.41E−05 |
| 9:6192209:C:T | C | 1.42E−06 |
| 9:6251507:G:T | G | 1.49E−05 |
| 9:6171224:C:T | T | 4.21E−05 |
| 9:6218346:G:A | A | 5.50E−05 |
| 9:6209755:A:G | A | 4.19E−05 |
| 9:6134926:C:T | T | 6.40E−05 |
| 9:6159193:T:C | T | 1.66E−05 |
| 9:6230912:T:C | T | 1.88E−05 |
| 9:6210099:T:C | T | 0.00013756 |
| 9:6288927:C:T | T | 2.56E−05 |
| 9:6229110:A:G | A | 3.97E−06 |
| 9:6143736:C:T | T | 8.75E−06 |
| 9:6225825:C:A | C | 8.15E−06 |
| 9:6285050:G:A | G | 4.05E−06 |
| 9:6191508:G:A | G | 5.58E−05 |
| 9:6226688:T:C | T | 3.57E−06 |
| 9:6270094:A:C | C | 3.20E−05 |
| 9:6176770:T:G | T | 3.65E−05 |
| 9:6234332:T:C | C | 2.69E−05 |
| 9:6243392:T:C | C | 7.28E−05 |
| 9:6279768:A:G | A | 5.77E−06 |
| 9:6224977:T:C | T | 3.91E−06 |
| 9:6150622:T:C | C | 6.62E−06 |
| 9:6252689:T:C | C | 3.15E−05 |
| 9:6154158:G:A | A | 2.43E−05 |
| 9:6281214:C:T | C | 6.10E−06 |
| 9:6222110:T:C | C | 3.06E−05 |
| 9:6229661:T:G | G | 3.20E−05 |
| 9:6246256:T:C | T | 1.55E−05 |
| 9:6255881:T:G | G | 1.60E−05 |
| 9:6131460:G:A | A | 6.38E−05 |
| 9:6234546:C:A | A | 1.26E−05 |
| 9:6129017:T:C | T | 2.68E−06 |
| 9:6175522:G:T | T | 4.62E−05 |
| 9:6263871:A:G | G | 3.00E−05 |
| 9:6200562:G:T | G | 3.56E−05 |
| 9:6254860:A:C | C | 3.02E−05 |
| 9:6280227:C:A | C | 2.81E−06 |
| 9:6240084:T:C | T | 1.11E−05 |
| 9:6172380:A:G | G | 0.00012136 |
| 9:6236501:T:G | T | 3.06E−05 |
| 9:6129637:A:C | C | 6.43E−05 |
| 9:6226427:A:G | G | 2.27E−05 |
| 9:6272766:C:A | A | 2.04E−05 |
| 9:6255010:A:C | C | 1.59E−05 |
| 9:6191110:T:C | T | 5.50E−05 |
| 9:6247693:G:A | G | 1.37E−05 |
| 9:6130940:A:G | G | 6.36E−05 |
| 9:6198578:A:C | C | 3.49E−05 |
| 9:6278071:A:G | A | 5.46E−06 |
| 9:6171731:T:C | T | 4.02E−05 |
| 9:6153708:C:A | C | 2.77E−05 |
| 9:6231455:C:T | T | 8.31E−05 |
| 9:6276216:C:T | T | 3.81E−05 |
| 9:6287142:C:A | A | 1.28E−05 |
| 9:6242950:C:T | T | 1.69E−05 |
| 9:6238750:A:C | C | 7.28E−05 |
| 9:6281660:G:A | G | 1.26E−05 |
| 9:6277740:G:A | G | 5.42E−06 |
| 9:6165405:C:T | T | 2.55E−05 |
| 9:6284665:A:G | G | 3.71E−05 |
| 9:6240953:A:C | C | 7.25E−05 |
| 9:6146441:G:T | T | 7.27E−05 |
| 9:6277534:A:G | A | 1.69E−05 |
| 9:6284901:G:A | A | 3.61E−05 |
| 9:6232985:T:C | C | 1.32E−05 |
| 9:6222302:T:G | G | 3.66E−05 |
| 9:6142674:G:A | G | 1.85E−06 |
| 9:6284723:T:C | T | 1.48E−05 |
| 9:6134750:A:G | G | 3.61E−05 |
| 9:6245931:C:T | T | 3.10E−05 |
| 9:6205394:T:C | T | 5.74E−05 |
| 9:6177291:G:T | G | 8.25E−05 |
| 9:6184145:G:A | A | 2.03E−05 |
| 9:6264768:G:T | T | 1.87E−05 |
| 9:6214050:T:G | T | 7.63E−05 |
| 9:6134642:G:A | A | 3.61E−05 |
| 9:6133014:C:T | T | 3.37E−05 |
| 9:6208893:T:C | C | 5.52E−05 |
| 9:6229417:G:A | G | 2.28E−05 |
| 9:6132621:A:G | G | 6.39E−05 |
| 9:6288871:G:A | A | 1.54E−05 |
| 9:6254694:A:C | C | 1.58E−05 |
| 9:6154012:G:A | A | 5.13E−05 |
| 9:6255319:G:A | G | 1.49E−05 |
| 9:6264168:T:C | C | 1.86E−05 |
| 9:6204469:G:A | G | 4.56E−05 |
| 9:6253571:C:T | C | 1.42E−05 |
| 9:6195285:C:T | C | 8.85E−05 |
| 9:6133074:C:T | T | 7.32E−06 |
| 9:6168335:T:C | C | 2.57E−05 |
| 9:6163823:G:A | A | 4.99E−05 |
| 9:6177302:C:T | T | 0.00012595 |
| 9:6154200:G:A | G | 0.00010329 |
| 9:6235753:G:T | T | 1.75E−05 |
| 9:6158026:A:G | A | 7.35E−05 |
| 9:6221246:T:C | T | 2.26E−05 |
| 9:6130520:T:C | C | 2.62E−05 |
| 9:6226295:C:T | C | 3.65E−06 |
| 9:6275456:T:C | T | 3.67E−06 |
| 9:6213468:C:T | T | 0.00012959 |
| 9:6291125:T:C | T | 7.91E−06 |
| 9:6243819:T:C | T | 1.10E−05 |
| 9:6200576:A:C | A | 6.85E−05 |
| 9:6226881:G:A | G | 7.62E−05 |
| 9:6233376:T:C | C | 7.38E−05 |
| 9:6277820:A:G | G | 1.40E−05 |
| 9:6240324:T:C | C | 7.76E−05 |
| 9:6173798:T:C | T | 4.87E−05 |
| 9:6246144:G:A | G | 1.52E−05 |
| 9:6237186:T:C | C | 3.85E−05 |
| 9:6214036:G:A | G | 4.24E−05 |
| 9:6288724:G:A | A | 1.53E−05 |
| 9:6160648:C:T | T | 4.77E−05 |
| 9:6197392:T:C | T | 0.00013717 |
| 9:6285589:G:A | A | 1.57E−05 |
| 9:6169383:G:A | G | 2.19E−05 |
| 9:6144333:C:T | T | 3.91E−05 |
| 9:6231318:A:C | A | 4.91E−05 |
| 9:6133663:C:A | A | 3.14E−05 |
| 9:6201001:T:C | T | 3.48E−05 |
| 9:6280786:A:G | A | 5.94E−06 |
| 9:6235009:A:G | G | 7.76E−05 |
| 9:6282511:G:A | A | 0.00021266 |
| 9:6251012:C:A | C | 2.04E−05 |
| 9:6266244:A:G | G | 3.09E−05 |
| 9:6177677:G:A | G | 2.97E−05 |
| 9:6216138:A:G | A | 3.04E−05 |
| 9:6251455:T:C | C | 1.73E−05 |
| 9:6248779:G:A | A | 7.02E−06 |
| 9:6240235:T:G | G | 7.59E−05 |
| 9:6130938:A:G | G | 6.36E−05 |
| 9:6237547:A:C | C | 7.44E−05 |
| 9:6253301:T:C | T | 1.79E−05 |
| 9:6192650:C:A | C | 5.12E−05 |
| 9:6138915:C:T | C | 4.20E−05 |
| 9:6248035:C:T | C | 1.59E−05 |
| 9:6135481:T:C | T | 7.62E−05 |
| 9:6170162:T:G | G | 2.48E−05 |
| 9:6288823:G:A | G | 4.15E−06 |
| 9:6213148:A:G | A | 0.00012898 |
| 9:6254900:G:A | A | 1.59E−05 |
| 9:6248457:C:T | C | 6.77E−05 |
| 9:6251588:T:C | T | 1.50E−05 |
| 9:6236407:T:G | T | 2.05E−05 |
| 9:6257054:T:C | C | 2.87E−05 |
| 9:6257898:A:G | A | 3.67E−06 |
| 9:6187132:C:A | A | 0.00020186 |
| 9:6133656:G:A | A | 2.85E−05 |
| 9:6197547:G:A | G | 2.26E−05 |
| 9:6229662:A:C | C | 3.14E−05 |
| 9:6155226:G:T | T | 4.77E−05 |
| 9:6134466:C:A | A | 2.99E−06 |
| 9:6210107:A:C | A | 4.16E−05 |
| 9:6228694:A:G | A | 3.55E−06 |
| 9:6283836:C:A | A | 1.53E−05 |
| 9:6277317:G:A | G | 4.87E−06 |
| 9:6226086:C:T | C | 9.29E−06 |
| 9:6287726:C:A | A | 1.53E−05 |
| 9:6266440:A:G | A | 4.57E−06 |
| 9:6275720:T:C | C | 1.49E−05 |
| 9:6236350:G:A | A | 1.65E−05 |
| 9:6199536:T:C | T | 5.82E−05 |
| 9:6218595:C:T | C | 3.29E−05 |
| 9:6256678:T:C | C | 3.05E−05 |
| 9:6274892:T:C | C | 1.58E−05 |
| 9:6225535:A:G | A | 3.75E−06 |
| 9:6222149:T:C | T | 5.22E−05 |
| 9:6190076:A:C | C | 0.00012164 |
| 9:6260683:T:C | C | 2.94E−05 |
| 9:6226207:C:T | C | 3.64E−06 |
| 9:6164771:C:A | A | 2.51E−05 |
| 9:6166769:G:A | A | 2.84E−05 |
| 9:6167452:G:A | A | 7.54E−05 |
| 9:6135571:C:T | T | 1.52E−05 |
| 9:6228228:T:C | C | 3.31E−05 |
| 9:6248408:G:A | G | 1.41E−05 |
| 9:6253297:C:T | C | 1.41E−05 |
| 9:6134402:C:T | T | 3.61E−05 |
| 9:6189633:C:T | C | 1.57E−05 |
| 9:6134212:A:G | G | 3.93E−05 |
| 9:6144065:G:A | G | 8.11E−06 |
| 9:6146800:T:C | C | 5.65E−05 |
| 9:6146121:C:T | T | 5.63E−05 |
| 9:6151129:T:C | T | 1.54E−05 |
| 2:102293872:C:T | T | 6.00E−05 |
| 2:102314421:A:G | G | 1.30E−05 |
| 2:102351896:C:T | C | 5.97E−05 |
| 2:102321022:G:A | A | 1.26E−05 |
| 2:102349607:G:A | A | 5.72E−05 |
| 2:102334613:A:G | G | 1.31E−05 |
| 2:102319514:G:A | A | 4.64E−05 |
| 2:102332479:A:G | G | 1.28E−05 |
| 2:102344025:A:G | G | 7.00E−05 |
| 2:102303125:C:T | T | 7.67E−05 |
| 2:102316052:G:T | G | 0.00012327 |
| 2:102353524:C:A | C | 6.17E−05 |
| 2:102354740:T:C | T | 6.18E−05 |
| 2:102302845:T:C | C | 1.36E−05 |
| 2:102306070:C:T | T | 2.09E−06 |
| 2:102313009:G:A | A | 1.37E−05 |
| 2:102353783:T:G | T | 6.15E−05 |
| 2:102350330:C:A | C | 0.00011817 |
| 2:102308202:C:T | T | 1.36E−05 |
| 2:102305323:T:G | G | 1.45E−05 |
| 2:102289177:G:A | A | 5.95E−05 |
| 2:102341256:C:T | T | 7.49E−05 |
| 2:102339400:G:T | T | 4.51E−05 |
| 2:102295714:C:T | T | 5.12E−05 |
| 2:102344906:T:C | C | 5.26E−05 |
| 2:102341072:T:C | C | 4.53E−05 |
| 2:102313886:G:A | A | 7.64E−05 |
| 2:102304806:A:C | C | 7.63E−05 |
| 2:102315787:C:T | T | 1.36E−05 |
| 2:102334602:C:A | C | 1.25E−05 |
| 2:102311189:A:G | A | 1.18E−05 |
| 2:102339223:G:T | T | 4.51E−05 |
| 2:102350776:T:C | T | 6.14E−05 |
| 2:102310051:T:C | C | 1.40E−05 |
| 2:102312157:G:A | G | 0.00012438 |
| 2:102297036:T:G | G | 7.45E−06 |
| 2:102300289:G:A | G | 1.42E−05 |
| 2:102330217:A:G | G | 4.67E−05 |
| 2:102353347:C:T | T | 4.53E−05 |
| 2:102354353:T:C | T | 6.00E−05 |
| 2:102293987:G:A | G | 1.50E−05 |
| 2:102294597:C:T | T | 7.15E−06 |
| 2:102332701:C:T | T | 7.70E−05 |
| 2:102330172:G:T | T | 7.59E−05 |
| 2:102295601:A:G | G | 6.19E−05 |
| 2:102337432:T:G | G | 7.45E−05 |
| 2:102308155:C:T | C | 0.00012353 |
| 2:102316102:C:A | A | 7.77E−05 |
| 2:102291170:T:C | T | 6.79E−06 |
| 2:102315090:G:T | T | 1.30E−05 |
| 2:102297364:C:T | T | 6.12E−05 |
| 2:102345469:C:T | T | 5.27E−05 |
| 2:102353829:A:G | A | 6.15E−05 |
| 2:102313405:G:A | A | 7.60E−05 |
| 2:102324755:T:C | C | 1.29E−05 |
| 2:102355204:A:G | A | 6.17E−05 |
| 2:102350953:T:C | T | 6.14E−05 |
| 2:102299111:A:G | A | 5.07E−06 |
| 2:102299202:T:C | T | 1.54E−05 |
| 2:102293120:G:A | A | 6.13E−05 |
| 2:102313847:A:G | G | 1.31E−05 |
| 2:102310480:T:C | C | 7.62E−05 |
| 2:102315074:C:T | C | 0.00012056 |
| 2:102342800:T:C | C | 5.02E−05 |
| 2:102338622:G:A | G | 0.00012408 |
| 2:102289716:C:T | T | 5.71E−05 |
| 2:102334362:A:C | C | 7.72E−05 |
| 2:102308975:C:T | C | 0.00012354 |
| 2:102321875:C:T | T | 1.28E−05 |
| 2:102291820:G:A | A | 6.13E−05 |
| 2:102321506:G:A | A | 7.60E−05 |
| 2:102300779:C:T | C | 0.00012232 |
| 2:102335391:G:A | A | 1.90E−06 |
| 2:102300842:C:A | C | 1.46E−05 |
| 2:102298194:A:G | G | 2.78E−05 |
| 2:102351751:C:A | C | 6.13E−05 |
| 2:102327786:T:G | T | 0.00012541 |
| 2:102337753:G:A | G | 0.00012417 |
| 2:102309801:C:T | T | 1.95E−06 |
| 2:102298489:G:A | A | 7.14E−06 |
| 2:102344364:A:G | A | 7.40E−05 |
| 2:102353453:C:T | C | 6.17E−05 |
| 2:102349850:G:A | G | 7.47E−05 |
| 2:102322538:C:T | T | 7.57E−05 |
| 2:102310626:G:A | A | 1.32E−05 |
| 2:102354217:T:C | T | 1.09E−05 |
| 2:102292333:C:T | C | 7.34E−07 |
| 2:102321929:T:C | C | 1.91E−06 |
| 2:102317342:G:A | A | 1.30E−05 |
| 2:102303577:T:C | C | 1.42E−05 |
| 2:102351384:C:A | A | 6.78E−05 |
| 2:102335900:G:A | A | 4.58E−05 |
| 2:102350971:G:A | G | 6.17E−05 |
| 2:102313687:G:A | A | 1.31E−05 |
| 2:102351547:G:A | G | 6.09E−05 |
| 2:102341776:C:T | C | 9.43E−06 |
| 2:102351825:T:C | T | 6.15E−05 |
| 2:102332172:A:C | A | 7.47E−07 |
| 2:102351752:A:G | A | 6.17E−05 |
| 2:102295809:C:T | T | 7.60E−06 |
| 2:102328661:C:A | A | 2.66E−05 |
| 2:102315193:G:T | G | 1.09E−05 |
| 2:102300536:C:T | T | 2.00E−06 |
| 2:102310521:G:A | A | 1.33E−05 |
| 2:102331721:A:G | G | 7.60E−05 |
| 2:102332359:C:T | T | 1.28E−05 |
| 2:102348693:C:T | T | 9.35E−07 |
| 2:102337157:G:T | G | 0.00012358 |
| 2:102354290:T:C | T | 6.04E−05 |
| 2:102304870:G:A | A | 2.31E−06 |
| 2:102351127:A:C | A | 6.15E−05 |
| 2:102346279:G:A | A | 5.25E−05 |
| 2:102348932:A:C | C | 5.23E−05 |
| 2:102324878:T:C | C | 1.47E−05 |
| 2:102340831:A:G | G | 0.00010073 |
| 2:102300505:T:C | C | 1.48E−06 |
| 2:102354846:C:A | C | 6.18E−05 |
| 2:102315466:C:T | T | 1.31E−05 |
| 2:102288891:A:G | G | 5.97E−05 |
| 2:102334200:A:G | G | 7.73E−06 |
| 2:102350970:T:C | T | 6.15E−05 |
| 2:102314862:G:A | G | 9.01E−05 |
| 2:102322974:T:C | C | 1.30E−05 |
| 2:102309902:G:A | A | 7.82E−05 |
| 2:102312552:T:C | C | 1.31E−05 |
| 2:102347489:T:C | C | 5.25E−05 |
| 2:102297131:C:T | T | 7.69E−06 |
| 2:102298752:A:G | G | 2.75E−06 |
| 2:102314197:T:C | C | 1.32E−05 |
| 2:102317092:C:T | T | 1.31E−05 |
| 2:102294000:T:C | C | 4.47E−06 |
| 2:102351615:T:C | T | 6.15E−05 |
| 2:102300783:C:T | C | 1.46E−05 |
| 2:102290432:G:A | G | 7.49E−07 |
| 2:102297754:A:G | A | 0.0001228 |
| 2:102344124:C:A | A | 7.01E−05 |
| 2:102343186:A:G | G | 1.89E−06 |
| 2:102339008:C:A | C | 3.46E−05 |
| 2:102321084:C:T | C | 0.00012385 |
| 2:102292817:T:C | C | 3.56E−06 |
| 2:102308089:C:T | T | 1.35E−05 |
| 2:102318514:C:T | C | 0.00012361 |
| 2:102313036:T:C | T | 1.14E−05 |
| 2:102321968:A:G | G | 1.04E−05 |
| 2:102337916:G:A | G | 9.29E−06 |
| 2:102351902:T:C | T | 6.17E−05 |
| 2:102296204:C:A | A | 2.20E−06 |
| 2:102323373:C:T | T | 1.29E−05 |
| 2:102355662:G:A | A | 7.86E−06 |
| 2:102332010:G:A | A | 7.70E−05 |
| 2:102298109:T:C | C | 7.12E−06 |
| 2:102320521:G:A | A | 1.26E−05 |
| 2:102354903:G:T | T | 5.21E−05 |
| 2:102351468:A:G | A | 6.15E−05 |
| 2:102324792:C:T | T | 1.57E−06 |
| 2:102353489:C:T | C | 6.20E−05 |
| 2:102324851:G:A | G | 0.00012424 |
| 2:102317298:A:G | G | 1.31E−05 |
| 2:102348401:C:T | T | 4.55E−05 |
| 2:102316354:G:A | G | 1.41E−05 |
| 2:102337730:T:C | C | 1.14E−05 |
| 2:102331944:T:C | T | 2.43E−05 |
| 2:102311266:A:G | A | 1.08E−06 |
| 2:102354347:A:G | A | 1.14E−05 |
| 2:102313920:A:G | G | 1.31E−05 |
| 2:102322576:C:T | T | 7.69E−05 |
| 2:102340888:C:T | C | 0.00012426 |
| 2:102336338:T:C | C | 7.46E−05 |
| 2:102339802:A:G | G | 7.44E−05 |
| 2:102342620:G:A | A | 7.40E−05 |
| 2:102338193:C:T | C | 0.00012402 |
| 2:102355405:G:T | T | 7.81E−07 |
| 2:102351398:T:C | T | 6.15E−05 |
| 2:102315342:A:G | G | 1.38E−05 |
| 2:102309906:G:A | A | 4.75E−05 |
| 2:102328781:G:A | A | 1.25E−05 |
| 2:102353511:C:A | C | 6.17E−05 |
| 2:102323952:T:C | C | 1.30E−05 |
| 2:102314488:A:G | G | 1.37E−05 |
| 2:102315833:C:T | T | 1.36E−05 |
| 2:102314935:T:C | C | 1.31E−05 |
| 2:102315152:T:C | T | 0.00012348 |
| 2:102344027:T:C | C | 5.29E−05 |
| 2:102344359:T:C | C | 5.26E−05 |
| 2:102334722:A:G | A | 1.46E−05 |
| 2:102349411:C:T | T | 4.56E−05 |
| 2:102300774:T:C | C | 1.35E−05 |
| 2:102308224:G:A | G | 1.47E−05 |
| 2:102356074:C:T | C | 1.22E−05 |
| 2:102304770:G:A | A | 9.38E−05 |
| 2:102348282:C:T | T | 6.91E−05 |
| 2:102343547:G:A | A | 4.66E−05 |
| 2:102335757:T:C | C | 7.46E−05 |
| 2:102350089:A:G | A | 0.00012386 |
| 2:102354705:C:T | T | 4.55E−05 |
| 2:102355021:C:A | C | 6.17E−05 |
| 2:102347168:G:A | A | 5.27E−05 |
| 2:102351397:C:T | C | 6.14E−05 |
| 2:102345890:T:C | C | 5.25E−05 |
| 2:102309860:T:C | C | 1.34E−05 |
| 2:102336984:G:A | G | 1.05E−05 |
| 2:102319345:C:A | A | 1.27E−05 |
| 2:102305996:A:G | G | 7.62E−05 |
| 2:102313888:T:G | G | 7.63E−05 |
| 2:102352210:T:C | C | 1.52E−06 |
| 2:102356347:G:A | A | 1.38E−06 |
| 2:102332937:G:A | A | 0.00010382 |
| 2:102354299:T:C | T | 6.17E−05 |
| 2:102335017:A:G | A | 1.42E−05 |
| 2:102353645:A:G | A | 6.15E−05 |
| 2:102321423:C:T | C | 0.00012368 |
| 2:102336607:T:C | C | 1.14E−05 |
| 2:102318777:T:C | C | 1.29E−05 |
| 2:102301328:T:C | C | 1.36E−05 |
| 2:102350323:T:C | C | 1.43E−06 |
| 2:102324577:G:A | G | 0.00012362 |
| 2:102301558:G:A | G | 1.35E−06 |
| 2:102301274:C:T | T | 1.91E−05 |

In some embodiments, the genetic variants are chosen from any one or more of the variants listed in Table 2, which is a subset of the variants from Table 1 (variant id is according to GRCh38/hg38 human genome assembly coordinates).

TABLE 2

| variant_id | effective_allele |
|---|---|
| 2:102350089:A:G | A |
| 9:6187132:C:A | A |
| 2:102309902:G:A | A |
| 9:6209697:A:G | A |

Risk assessments using large numbers of genetic variants offers the advantage of increased predictive power. In some embodiments, one or more of the genetic variants is a single nucleotide polymorphism (SNP). In some embodiments, one or more of the genetic variants is an insertion. In some embodiments, one or more of the genetic variants is a deletion. In some embodiments, one or more of the genetic variants is a structural variant. In some embodiments, one or more of the genetic variants is a copy-number variation. In any of the embodiments described herein, the presence or absence, or amount thereof, of any of the genetic variants described herein can be replaced with the presence or absence of any particular mRNA molecule, or the expression level thereof.

In some embodiments, the present disclosure provides methods of determining an IL-33 Asthma PRS for a subject, the methods comprising identifying whether at least about 2 genetic variants, at least about 5 genetic variants, at least about 10 genetic variants, at least about 15 genetic variants, at least about 20 genetic variants, at least about 30 genetic variants, at least about 40 genetic variants, at least about 50 genetic variants, at least about 60 genetic variants, at least about 70 genetic variants, at least about 100 genetic variants, at least about 200 genetic variants, at least about 500 genetic variants, at least about 1,000 genetic variants, at least about 2,000 genetic variants, at least about 3,000 genetic variants, at least about 4,000 genetic variants, at least about 5,000 genetic variants, at least about 6,000 genetic variants, at least about 7,000 genetic variants, at least about 8,000 genetic variants, at least about 9,000 genetic variants, or at least about 10,000 genetic variants associated with a risk of developing asthma are present in a biological sample from the subject. The presence of a risk allele increases the subject's IL-33 Asthma PRS.

In some embodiments, the disclosure provides a method of determining an IL-33 Asthma PRS for a subject comprising identifying whether one or more genetic variants associated with a risk of developing asthma are present in a biological sample from the subject and calculating an IL-33 Asthma PRS for the subject based on the identified genetic variants, wherein the IL-33 Asthma PRS is calculated by aggregating, such as by summing, the risk score (or weighted risk score) associated with each identified genetic variant. The number of identified genetic variants can be at least about 2 genetic variants, at least about genetic variants, at least about 10 genetic variants, at least about 15 genetic variants, at least about 20 genetic variants, at least about 30 genetic variants, at least about 40 genetic variants, at least about 50 genetic variants, at least about 95 genetic variants, at least about 100 genetic variants, at least about 200 genetic variants, at least about 500 genetic variants, at least about 1,000 genetic variants, at least about 2,000 genetic variants, at least about 3,000 genetic variants, at least about 4,000 genetic variants, at least about 5,000 genetic variants, at least about 6,000 genetic variants, at least about 7,000 genetic variants, at least about 8,000 genetic variants, at least about 9,000 genetic variants, or at least about 10,000 genetic variants associated with a risk of developing asthma. In some embodiments, the disclosure provides methods of determining an IL-33 Asthma PRS for a subject comprising identifying whether the genetic variants associated with a risk of developing asthma are present in a biological sample from the subject, wherein the identification process comprises measuring the presence of the at least about 2 genetic variants, at least about 5 genetic variants, at least about 10 genetic variants, at least about 15 genetic variants, at least about 20 genetic variants, at least about 30 genetic variants, at least about 40 genetic variants, at least about 50 genetic variants, at least about 95 genetic variants, at least about 100 genetic variants, at least about 200 genetic variants, at least about 500 genetic variants, at least about 1,000 genetic variants, at least about 2,000 genetic variants, at least about 3,000 genetic variants, at least about 4,000 genetic variants, at least about 5,000 genetic variants, at least about 6,000 genetic variants, at least about 7,000 genetic variants, at least about 8,000 genetic variants, at least about 9,000 genetic variants, or at least about 10,000 genetic variants.

As an exemplary method, an IL-33 Asthma PRS can be determined from, for example, data obtained from a GWAS of disease risk. For example, in a representative hypothetical GWAS, a GWAS may have identified four genetic variants associated with a disease. Each of the genetic variants may be associated with one or more genes. A value, such as an Odds Ratio, can be calculated for each individual genetic variant. A particular subject's IL-33 Asthma PRS can be determined by multiplying the log value of the individual Odds Ratio for each variant by the Number Effect Alleles (which is the number of copies of the genetic variant in the genome; i.e., either 0, 1, or 2), and then summing the resultant values. This type of determination can be described by Table 3.

TABLE 3

| | | | | Number | |
|---|---|---|---|---|---|
| | Variant | Effect | Odds Ratio | Effect | Log(OR) × Number |
| Gene | rsID | Allele | (OR) | Alleles | Effect Alleles |
| A | rs000001 | T | 2.14 | 1 | 0.761 |
| B | rs000002 | A | 1.85 | 0 | 0.000 |
| C | rs000003 | A | 1.36 | 0 | 0.000 |
| . . . | . . . | . . . | . . . | . . . | . . . |
| D | rs000004 | C | 1.28 | 1 | 0.247 |

| | |
|---|---|
| Total Score | 10.910 |

Thus, the subject's IL-33 Asthma PRS is the sum of the individual values in the last column of the Table taking into consideration any number of genetic variants associated with the particular disease. This simplified methodology for determining a subject's IL-33 Asthma PRS is for exemplary purposes only and shall not be construed to be limiting in any manner. The IL-33 Asthma PRS in the above table is a weighted score because each genetic variant may carry a different weight depending on the particular Odds Ratio and the Number Effect Alleles value.

In some embodiments, the disclosure provides a method of assigning an asthma risk group to a subject comprising identifying whether the genetic variants are present in a biological sample from the subject, calculating an IL-33 Asthma PRS for the subject based on the identified genetic variants, and assigning the subject to a risk group based on the IL-33 Asthma PRS. The threshold PRSs can be determined by a hierarchy. In some embodiments, the hierarchy can be by percentiles. By way of a non-limiting example, the IL-33 Asthma PRS may be divided into quintiles, e.g., a top quintile, a top-intermediate quintile, an intermediate quintile, an intermediate-bottom quintile, and a bottom quintile, wherein the top quintile of IL-33 Asthma PRSs correspond the highest genetic risk group and the bottom quintile of IL-33 Asthma PRSs correspond to the lowest genetic risk group. The number of identified genetic variants can be at least about 2 genetic variants, at least about 5 genetic variants, at least about 10 genetic variants, at least about 15 genetic variants, at least about genetic variants, at least about 30 genetic variants, at least about 40 genetic variants, at least about 50 genetic variants, at least about 95 genetic variants, at least about 100 genetic variants, at least about 200 genetic variants, at least about 500 genetic variants, at least about 1,000 genetic variants, at least about 2,000 genetic variants, at least about 3,000 genetic variants, at least about 4,000 genetic variants, at least about 5,000 genetic variants, at least about 6,000 genetic variants, at least about 7,000 genetic variants, at least about 8,000 genetic variants, at least about 9,000 genetic variants, or at least about 10,000 genetic variants associated with asthma.

In some embodiments, the disclosure provides methods for selecting subjects or candidates for administration of an IL-33 antagonist, an interleukin-4 receptor alpha antagonist, and/or an interleukin-13 receptor antagonist comprising identifying whether at least about 2 genetic variants, at least about 5 genetic variants, at least about 10 genetic variants, at least about 15 genetic variants, at least about 20 genetic variants, at least about genetic variants, at least about 40 genetic variants, at least about 50 genetic variants, at least about 95 genetic variants, at least about 100 genetic variants, at least about 200 genetic variants, at least about 500 genetic variants, at least about 1,000 genetic variants, at least about 2,000 genetic variants, at least about 3,000 genetic variants, at least about 4,000 genetic variants, at least about 5,000 genetic variants, at least about 6,000 genetic variants, at least about 7,000 genetic variants, at least about 8,000 genetic variants, at least about 9,000 genetic variants, or at least about 10,000 genetic variants are present in a biological sample from the subject or candidate; calculating an IL-33 Asthma PRS for the subject or candidate based on the identified genetic variants; and selecting the subject or candidate for administration of an IL-33 antagonist, an interleukin-4 receptor alpha antagonist, and/or an interleukin-13 receptor antagonist.

In some embodiments, the disclosure provides methods for selecting a population of subjects or candidates for administration of an IL-33 antagonist, an interleukin-4 receptor alpha antagonist, and/or an interleukin-13 receptor antagonist, comprising identifying whether at least about 2 genetic variants, at least about 5 genetic variants, at least about 10 genetic variants, at least about 15 genetic variants, at least about 20 genetic variants, at least about 30 genetic variants, at least about 40 genetic variants, at least about 50 genetic variants, at least about 95 genetic variants, at least about 100 genetic variants, at least about 200 genetic variants, at least about 500 genetic variants, at least about 1,000 genetic variants, at least about 2,000 genetic variants, at least about 3,000 genetic variants, at least about 4,000 genetic variants, at least about 5,000 genetic variants, at least about 6,000 genetic variants, at least about 7,000 genetic variants, at least about 8,000 genetic variants, at least about 9,000 genetic variants, or at least about 10,000 genetic variants associated with asthma are present in a biological sample from each subject or candidate of the population of subjects or candidates; calculating an IL-33 Asthma PRS for each subject or candidate based on the identified genetic variants; and selecting the subjects or candidates for administration of an IL-33 antagonist, an interleukin-4 receptor alpha antagonist, and/or an interleukin-13 receptor antagonist.

In some embodiments, the number of identified genetic variants is at least 4 genetic variants associated with asthma. In some embodiments, the number of identified genetic variants is at least 5 genetic variants associated with asthma. In some embodiments, the number of identified genetic variants is at least 10 genetic variants associated with asthma. In some embodiments, the number of identified genetic variants is at least 20 genetic variants associated with asthma. In some embodiments, the number of identified genetic variants is at least 30 genetic variants associated with asthma. In some embodiments, the number of identified genetic variants is at least 40 genetic variants associated with asthma. In some embodiments, the number of identified genetic variants is at least 50 genetic variants associated with asthma. In some embodiments, the number of identified genetic variants is at least 70 genetic variants associated with asthma. In some embodiments, the number of identified genetic variants is at least 100 genetic variants associated with asthma. In some embodiments, the number of identified genetic variants is at least 500 genetic variants associated with asthma. In some embodiments, the number of identified genetic variants is at least 1,000 genetic variants associated with asthma. In some embodiments, the number of identified genetic variants is at least 2,000 genetic variants associated with asthma. In some embodiments, the number of identified genetic variants is at least 3,000 genetic variants associated with asthma. In some embodiments, the number of identified genetic variants is at least 4,000 genetic variants associated with asthma. In some embodiments, the number of identified genetic variants is at least 5,000 genetic variants associated with asthma. In some embodiments, the number of identified genetic variants is at least 6,000 genetic variants associated with asthma. In some embodiments, the number of identified genetic variants is at least 7,000 genetic variants associated with asthma. In some embodiments, the number of identified genetic variants is at least 8,000 genetic variants associated with asthma. In some embodiments, the number of identified genetic variants is at least 9,000 genetic variants associated with asthma. In some embodiments, the number of identified genetic variants is at least 10,000 genetic variants associated with asthma.

In some embodiments, risk assessments comprise the highest weighted IL-33 Asthma PRS scores, including, but not limited to the top 50%, 55%, 60%, 70%, 80%, 90%, or 95% of IL-33 Asthma PRS scores from a subject population. In some embodiments of the disclosure, the threshold PRS is a value within the top 50%, 55%, 60%, 70%, 80%, 90%, or 95% percentile of the PRS value. In some embodiments of the disclosure, the threshold PRS is a value within the top 50% percentile of the PRS value. In some embodiments of the disclosure, the threshold PRS is a value within the top 55% percentile of the PRS value. In some embodiments of the disclosure, the threshold PRS is a value within the top 60% percentile of the PRS value. In some embodiments of the disclosure, the threshold PRS is a value within the top 65% percentile of the PRS value. In some embodiments of the disclosure, the threshold PRS is a value within the top 70% percentile of the PRS value. In some embodiments of the disclosure, the threshold PRS is a value within the top 75% percentile of the PRS value. In some embodiments of the disclosure, the threshold PRS is a value within the top 80% percentile of the PRS value. In some embodiments of the disclosure, the threshold PRS is a value within the top 85% percentile of the PRS value. In some embodiments of the disclosure, the threshold PRS is a value within the top 90% percentile of the PRS value. In some embodiments of the disclosure, the threshold PRS is a value within the top 95% percentile of the PRS value.

In some embodiments, the identified genetic variants comprise the highest risk genetic variants or genetic variants with a weighted risk score in the top 10%, top 20%, top 30%, top 40%, or top 50%. In some embodiments, the identified genetic variants comprise the highest risk genetic variants or genetic variants with a weighted risk score in the top 10%. In some embodiments, the identified genetic variants comprise the highest risk genetic variants or genetic variants with a weighted risk score in the top 20%. In some embodiments, the identified genetic variants comprise the highest risk genetic variants or genetic variants with a weighted risk score in the top 30%. In some embodiments, the identified genetic variants comprise the highest risk genetic variants or genetic variants with a weighted risk score in the top 40%. In some embodiments, the identified genetic variants comprise the highest risk genetic variants or genetic variants with a weighted risk score in the top 50%.

In some embodiments, the identified genetic variants comprise the genetic variants having association with asthma in the top 10%, top 20%, top 30%, top 40%, or top 50% of a p-value range. In some embodiments, the identified genetic variants comprise the genetic variants having association with asthma in the top 10% of a p-value range. In some embodiments, the identified genetic variants comprise the genetic variants having association with asthma in the top 20% of a p-value range. In some embodiments, the identified genetic variants comprise the genetic variants having association with asthma in the top 30% of a p-value range. In some embodiments, the identified genetic variants comprise the genetic variants having association with asthma in the top 40% of a p-value range. In some embodiments, the identified genetic variants comprise the genetic variants having association with asthma in the top 50% of a p-value range.

In some embodiments, each of the identified genetic variants comprise genetic variants having association with the IL-33 gene with a p-value of not larger than about 10⁻¹, about 10⁻², about 10⁻³, about 10⁻⁴, about 10⁻⁵, about 10⁻⁶, about 10⁻⁷ 10⁻⁸, about 10⁻⁹, about 10⁻¹⁰, about 10⁻¹¹, about 10⁻¹², about 10⁻¹³, about 10⁻¹⁴, or about 10⁻¹⁵. In some embodiments, the identified genetic variants comprise the genetic variants having association with the IL-33 gene with p-value of less than 5×10⁻⁸.

In some embodiments, the identified genetic variants comprise genetic variants having association with the IL-33 gene in high-risk subjects as compared to the rest of the reference population with an odds ratio (OR) of about 1.0 or greater, about 1.5 or greater, about 1.75 or greater, about 2.0 or greater, about 2.25 or greater, or about 2.75 or greater for the top half (up to 50%) of the distribution; about 1.5 or greater, about 1.75 or greater, about 2.0 or greater, about 2.25 or greater, about 2.5 or greater, or about 2.75 or greater of the top quarter (up to 55%) of the distribution; about 1.0 or greater, about 1.5 or greater, about 1.75 or greater, about 2.0 or greater, about 2.25 or greater, or about 2.75 or greater for up to 60% of the distribution; about 1.0 or greater, about 1.5 or greater, about 1.75 or greater, about 2.0 or greater, about 2.25 or greater, or about 2.75 or greater for up to 70% of the distribution; about 1.0 or greater, about 1.5 or greater, about 1.75 or greater, about 2.0 or greater, about 2.25 or greater, or about 2.75 or greater for up to 80% of the distribution; about 1.0 or greater, about 1.5 or greater, about 1.75 or greater, about 2.0 or greater, about 2.25 or greater, or about 2.75 or greater for up to 90% of the distribution; or about 1.0 or greater, about 1.5 or greater, about 1.75 or greater, about 2.0 or greater, about 2.25 or greater, or about 2.75 or greater for up to 95% of the distribution. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, or from about 6.5 to about 7.0. In some embodiments, high-risk subjects comprise subjects having IL-33 Asthma PRS scores in the top decile, quintile, or tertile of a reference population.

In some embodiments, the identified genetic variants comprise genetic variants having the highest genetic variant performance in the reference population. In some embodiments, genetic variant performance is calculated with respect to asthma risk based on statistical significance, strength of association, and/or a probability distribution.

In some embodiments, genetic variant scores are calculated using any PRS calculation methodology. In some embodiments, genetic variant scores are calculated using PRS calculation methodologies such as the LDpred method (or variations and/or versions thereof). LDpred is a Bayesian approach to calculate a posterior mean effect for all variants based on a prior (effect size in the prior genome-wide association study) and subsequent shrinkage based on linkage disequilibrium. LDpred creates a PRS using genome-wide variation with weights derived from a set of genome-wide association study (GWAS) summary statistics. See, VilhjáInnsson et al., Am. J. Hum. Genet., 2015, 97, 576-92. In some embodiments, alternate approaches for calculating genetic variant scores may be used, including SBayesR (Lloyd-Jones, LR, world wide web at “biorxiv.org/content/biorxiv/early/2019/01/17/522961.full.pdf”), Pruning and Thresholding (P&T) (Purcell, Nature, 2009, 460, 748-752), and conditional and joint analyses (COJO) (Yang et al., Nat. Genet., 2012, 44, 369-375). SBayesR is a Bayesian approach is similar to LDpred but allows for more flexibility in the posterior mean effects. Pruning and Thresholding (P&T) requires that a minimum p-value threshold (p-value associated with the variant from the source data file) and r2 threshold (measure of linkage disequilibrium (LD)) between variants be specified. P&T identifies the variant with the smallest p-value in each region and then “clumps” under that variant all other variants in the region with an r2 value that is larger than the specified r2. In the PRS, the index variant represents all the variants in the clump (only the index variant is included in the PRS with all other variants are excluded). COJO is similar conceptually to P&T but incorporates additional variants in a given LD block into the score if they demonstrate independent contribution to disease risk after conditioning on the index variant.

In some embodiments, genetic variant performance is calculated using the LDpred method, wherein the p value is from about 0.0001 to about 0.5. In some embodiments, genetic variant performance is calculated using the LDpred method, wherein the p value is about 0.5. In some embodiments, genetic variant performance is calculated using the LDpred method, wherein the p value is about 0.1. In some embodiments, genetic variant performance is calculated using the LDpred method, wherein the p value is about 0.05. In some embodiments, genetic variant performance is calculated using the LDpred method, wherein the p value is about 0.01. In some embodiments, genetic variant performance is calculated using the LDpred method, wherein the p value is about 0.005. In some embodiments, genetic variant performance is calculated using the LDpred method, wherein the p value is about 0.001. In some embodiments, genetic variant performance is calculated using the LDpred method, wherein the p value is about 0.0005. In some embodiments, genetic variant performance is calculated using the LDpred method, wherein the p value is about 0.0001.

In some embodiments, the method further comprises an initial step of obtaining a biological sample from the subject.

As used herein, a “biological sample” may contain whole cells, live cells and/or cell debris. The biological sample may contain (or be derived from) a “bodily fluid”. The present disclosure encompasses embodiments wherein the bodily fluid is selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammalian organism, for example by venipuncture, or other collecting or sampling procedures.

In any of the embodiments described herein, an endpoint of the IL-33 Asthma PRS can be an increased annual exacerbation rate. In any of the embodiments described herein, an endpoint of the IL-33 Asthma PRS can be a loss of asthma control. In any of the embodiments described herein, an endpoint of the IL-33 Asthma PRS can be Asthma Control Questionnaire-5 (ACQ-5).

The present disclosure provides methods of treating a subject having asthma or at risk of developing asthma, the methods comprising: administering an IL-33 antagonist to the subject when the subject's IL-33 asthma polygenic risk score (IL-33 Asthma PRS) is greater than or equal to a threshold IL-33 Asthma PRS, or administering an interleukin-4 receptor alpha antagonist and/or an interleukin-13 receptor antagonist to the subject when the subject's IL-33 Asthma PRS is less than a threshold IL-33 Asthma PRS, wherein the IL-33 Asthma PRS comprises a weighted aggregate of a plurality of genetic variants near or in the IL-33 and IL1RL1 genes associated with asthma.

The present disclosure also provides methods of treating a subject having asthma or at risk of developing asthma, the methods comprising: administering an interleukin-4 receptor alpha antagonist and/or an interleukin-13 receptor antagonist to the subject when the subject's IL-33 Asthma PRS is greater than or equal to a threshold IL-33 Asthma PRS and administering a standard amount or greater of a composition for treating asthma exacerbation, wherein the IL-33 Asthma PRS comprises a weighted aggregate of a plurality of genetic variants near or in the IL-33 and IL1RL1 genes associated with asthma.

The present disclosure also provides methods of determining whether a subject should be administered an interleukin-4 receptor alpha antagonist, an interleukin-13 receptor antagonist, and/or an interleukin-33 (IL-33) antagonist for treatment of asthma (or methods of classifying a subject having asthma; or methods of selecting a subject having asthma for treatment with an interleukin-4 receptor alpha antagonist/interleukin-13 receptor antagonist or treatment with an anti-IL-33 receptor antagonist; or methods of improving asthma treatment efficacy), the method comprising: determining or having determined the subject's IL-33 asthma Polygenic Risk Score (IL-33 Asthma PRS), wherein the IL-33 Asthma PRS comprises a weighted aggregate of a plurality of genetic variants near or in the IL-33 and IL1RL1 genes associated with asthma; and determining or having determined whether the subject's IL-33 Asthma PRS is greater than or equal to a threshold IL-33 Asthma PRS; and when the subject's IL-33 Asthma PRS is greater than or equal to the threshold IL-33 Asthma PRS, the subject should be administered the IL-33 antagonist; or when the subject's IL-33 Asthma PRS is less than the threshold IL-33 Asthma PRS, the subject should be administered the interleukin-4 receptor alpha antagonist and/or the interleukin-13 receptor antagonist. In some embodiments, the methods further comprise prescribing an interleukin-4 receptor alpha antagonist, an interleukin-13 receptor antagonist, and/or an interleukin-33 (IL-33) antagonist.

The present disclosure also provides methods of determining whether a subject having asthma should be administered a standard amount or greater of a composition for treating asthma exacerbation (or methods of classifying a subject having asthma; or methods of selecting a subject having asthma for treatment with an interleukin-4 receptor alpha antagonist/interleukin-13 receptor antagonist; or methods of improving asthma treatment efficacy), comprising: determining or having determined the subject's IL-33 asthma Polygenic Risk Score (IL-33 Asthma PRS), wherein the IL-33 Asthma PRS comprises a weighted aggregate of a plurality of genetic variants near or in the IL-33 and IL1RL1 genes associated with asthma; and when the subject's IL-33 Asthma PRS is greater than or equal to a threshold IL-33 Asthma PRS, the subject should be administered an interleukin-4 receptor alpha antagonist and/or an interleukin-13 receptor antagonist and a standard amount or greater of a composition for treating asthma exacerbation. In some embodiments, the methods further comprise prescribing an interleukin-4 receptor alpha antagonist and/or an interleukin-13 receptor antagonist.

The present disclosure also provides methods of determining whether a subject having asthma should be administered a standard amount or greater of a composition for treating a loss of asthma control (or methods of classifying a subject having a loss of asthma control; or methods of selecting a subject having a loss of asthma control for treatment with a composition for treating a loss of asthma control; or methods of improving asthma treatment efficacy), the method comprising: determining or having determined the subject's IL-33 asthma polygenic risk score (IL-33 Asthma PRS), wherein the IL-33 Asthma PRS comprises a weighted aggregate of a plurality of genetic variants near or in the IL-33 and IL1RL1 genes associated with asthma; and when the subject's IL-33 Asthma PRS is greater than or equal to a threshold IL-33 Asthma PRS, the subject should be administered a standard amount or greater of a composition for treating a loss of asthma control. In some embodiments, the methods further comprise prescribing a composition for treating a loss of asthma control.

The present disclosure also provides methods of determining whether a subject having asthma should be administered a standard amount or greater of a composition for treating asthma (or methods of classifying a subject having asthma; or methods of selecting a subject having asthma for treatment with a composition for treating a loss of asthma control; or methods of improving asthma treatment efficacy), the method comprising: determining or having determined the subject's IL-33 asthma polygenic risk score (IL-33 Asthma PRS), wherein the IL-33 Asthma PRS comprises a weighted aggregate of a plurality of genetic variants near or in the IL-33 and IL1RL1 genes associated with asthma; and when the subject's IL-33 Asthma PRS is greater than or equal to a threshold IL-33 Asthma PRS, the subject should be administered a standard amount or greater of a composition for treating a loss of asthma control. In some embodiments, the methods further comprise prescribing a composition for treating a loss of asthma control.

The present disclosure also provides methods of determining whether a subject having a lung disease should be administered a standard amount or greater of a composition for decreasing an eosinophil count (or methods of classifying a subject having a lung disease; or methods of selecting a subject having a lung disease for treatment with a composition for decreasing an eosinophil count; or methods of improving lung disease treatment efficacy), the method comprising: determining or having determined the subject's IL-33 asthma polygenic risk score (IL-33 Asthma PRS), wherein the IL-33 Asthma PRS comprises a weighted aggregate of a plurality of genetic variants near or in the IL-33 and IL1RL1 genes associated with asthma; and when the subject's IL-33 Asthma PRS is greater than or equal to a threshold IL-33 Asthma PRS, the subject should be administered a composition for decreasing an eosinophil count. In some embodiments, the methods further comprise prescribing a composition for decreasing an eosinophil count.

The present disclosure also provides methods of determining whether a subject having a lung disease should be administered a standard amount or greater of a composition for treating a lung disease (or methods of classifying a subject having lung disease; or methods of selecting a subject having a lung disease for treatment with a composition for decreasing asthma risk; or methods of improving lung disease treatment efficacy), the method comprising: determining or having determined the subject's IL-33 asthma polygenic risk score (IL-33 Asthma PRS), wherein the IL-33 Asthma PRS comprises a weighted aggregate of a plurality of genetic variants near or in the IL-33 and IL1RL1 genes associated with asthma; and when the subject's IL-33 Asthma PRS is greater than or equal to a threshold IL-33 Asthma PRS, the subject should be administered a standard amount or greater of a composition for decreasing asthma risk. In some embodiments, the methods further comprise prescribing a composition for decreasing asthma risk.

The present disclosure also provides methods of determining whether a subject having a lung disease should be administered a standard amount or greater of a composition for increasing an FEV1 value (or methods of classifying a subject having a lung disease; or methods of selecting a subject having a lung disease for treatment with a composition for increasing the FEV1 value; or methods of improving lung disease treatment efficacy), the method comprising: determining or having determined the subject's IL-33 asthma polygenic risk score (IL-33 Asthma PRS), wherein the IL-33 Asthma PRS comprises a weighted aggregate of a plurality of genetic variants near or in the IL-33 and IL1RL1 genes associated with asthma; and when the subject's IL-33 Asthma PRS is greater than or equal to a threshold IL-33 Asthma PRS, the subject should be administered a standard amount or greater of a composition for increasing the FEV1 value. In some embodiments, the methods further comprise prescribing a composition for increasing the FEV1 value.

The present disclosure also provides methods of determining whether a subject having a lung disease should be administered a standard amount or greater of a composition for decreasing a baseline basophil count (or methods of classifying a subject having a lung disease; or methods of selecting a subject having a lung disease for treatment with a composition for decreasing a baseline basophil count; or methods of improving lung disease treatment efficacy), the method comprising: determining or having determined the subject's IL-33 asthma polygenic risk score (IL-33 Asthma PRS), wherein the IL-33 Asthma PRS comprises a weighted aggregate of a plurality of genetic variants near or in the IL-33 and IL1RL1 genes associated with asthma; and when the subject's IL-33 Asthma PRS is greater than or equal to a threshold IL-33 Asthma PRS, the subject should be administered a standard amount or greater of a composition for decreasing a baseline basophil count. In some embodiments, the methods further comprise prescribing a composition for decreasing a baseline basophil count.

The present disclosure also provides methods of determining whether a subject having a lung disease should be administered a standard amount or greater of a composition for increasing a forced vital capacity (FVC) value (or methods of classifying a subject having a lung disease; or methods of selecting a subject having a lung disease for treatment with a composition for increasing FVC value; or methods of improving lung disease treatment efficacy), the method comprising: determining or having determined the subject's IL-33 asthma polygenic risk score (IL-33 Asthma PRS), wherein the IL-33 Asthma PRS comprises a weighted aggregate of a plurality of genetic variants near or in the IL-33 and IL1RL1 genes associated with asthma; and when the subject's IL-33 Asthma PRS is greater than or equal to a threshold IL-33 Asthma PRS, the subject should be administered a standard amount or greater of a composition for increasing FVC value. In some embodiments, the methods further comprise prescribing a composition for increasing FVC value.

In some embodiments, any of the methods described herein can be used to select a population of subjects or candidates for clinical trials, e.g., a clinical trial to determine whether a particular treatment or treatment plan is effective against a lung disease, such as asthma or COPD. In some embodiments, the selected candidates or subjects are divided into subgroups based on the identified genetic variants for each subject or candidate, and the method is used to determine whether a particular treatment or treatment plan is effective for a subject having a particular genetic variant or a particular group of genetic variants. For example, the methods described herein can be employed to determine susceptibility of a population of subjects to a particular treatment or treatment plan, wherein the population of subjects is selected based on the genetic variants identified in the subjects.

In some embodiments, the method is used to select a population of subjects or candidates for clinical trials, e.g., a clinical trial to determine whether a particular treatment or treatment plan is effective against a lung disease, such as asthma or COPD. In some embodiments, the desired risk group is a population comprising high risk subjects or candidates. In some embodiments, the selected population of subjects or candidates are responders, i.e., the subjects or candidates are responsive to the treatment or treatment plan.

In some embodiments the subjects are selected based on IL-33 Asthma PRS alone. For example, if a subject or a candidate that has an IL-33 Asthma PRS above a pre-determined threshold, the subject is selected for initiating treatment or a candidate is included in the clinical trial. In some embodiments, the threshold for treatment initiation or clinical trial inclusion is determined in relative terms. For example, in some embodiments, the threshold IL-33 Asthma PRS score is the top 50% within a reference population. In some embodiments, the threshold IL-33 Asthma PRS score is the top 40% within a reference population. In some embodiments, the threshold IL-33 Asthma PRS score is the top 30% within a reference population. In some embodiments, the threshold IL-33 Asthma PRS score is the top 25% within a reference population. In some embodiments, the threshold IL-33 Asthma PRS score is the top 20% within a reference population. In some embodiments, the threshold IL-33 Asthma PRS score is the top 15% within a reference population. In some embodiments, the threshold IL-33 Asthma PRS score is the top 10% (decile) within a reference population. In some embodiments, the threshold IL-33 Asthma PRS score is the top 5% within a reference population.

In any of the embodiments described herein, a subject having an IL-33 Asthma PRS score in the top 5%, in the top 10%, in the top 15%, in the top 20%, in the top 25%, or in the top 30%, within a reference population can be administered any of the IL-33 antagonists described herein, such as itepekimab. In any of the embodiments described herein, a subject having an IL-33 Asthma PRS score in the bottom 5%, in the bottom 10%, in the bottom 15%, in the bottom 20%, in the bottom 25%, or in the bottom 30%, within a reference population can be administered any of the interleukin-4 receptor alpha antagonists and/or interleukin-13 receptor antagonists described herein, such as dupilumab. In any of the embodiments described herein, a subject having an IL-33 Asthma PRS score between the top 30% and the bottom 30% within a reference population can be administered a combination of any of the IL-33 antagonists described herein and any of the interleukin-4 receptor alpha antagonists and/or interleukin-13 receptor antagonists described herein.

In some embodiments, the reference population for determination of relative IL-33 Asthma PRS score is at least about 100 subjects. In some embodiments, the reference population for determination of relative IL-33 Asthma PRS score is at least about 200 subjects. In some embodiments, the reference population for determination of relative IL-33 Asthma PRS score is at least about 500 subjects. In some embodiments, the reference population for determination of relative IL-33 Asthma PRS score is at least about 1,000 subjects. In some embodiments, the reference population for determination of relative IL-33 Asthma PRS score is at least about 3,000 subjects. In some embodiments, the reference population for determination of relative IL-33 Asthma PRS score is at least about 5,000 subjects. In some embodiments, the reference population for determination of relative IL-33 Asthma PRS score is at least about 7,500 subjects. In some embodiments, the reference population for determination of relative IL-33 Asthma PRS score is at least about 10,000 subjects. In some embodiments, the reference population for determination of relative IL-33 Asthma PRS score is at least about 12,000 subjects. In some embodiments, the reference population for determination of relative IL-33 Asthma PRS score is at least about 15,000 subjects. In some embodiments, the reference population for determination of relative IL-33 Asthma PRS score is at least about 20,000 subjects. In some embodiments, the reference population for determination of relative IL-33 Asthma PRS score is at least about 30,000 subjects. In some embodiments, the reference population for determination of relative IL-33 Asthma PRS score is at least about 50,000 subjects. In some embodiments, the reference population for determination of relative IL-33 Asthma PRS score is at least about 70,000 subjects. In some embodiments, the reference population for determination of relative IL-33 Asthma PRS score is at least about 100,000 subjects. In some embodiments, the reference population for determination of relative IL-33 Asthma PRS score is at least about 200,000 subjects. In some embodiments, the reference population for determination of relative IL-33 Asthma PRS score is at least about 300,000 subjects. In some embodiments, the reference population for determination of relative IL-33 Asthma PRS score is at least about 400,000 subjects. In some embodiments, the reference population for determination of relative IL-33 Asthma PRS score is at least about 500,000 subjects. In some embodiments, the reference population for determination of relative IL-33 Asthma PRS score is at least about 600,000 subjects. In some embodiments, the reference population for determination of relative IL-33 Asthma PRS score is at least about 700,000 subjects. In some embodiments, the reference population for determination of relative IL-33 Asthma PRS score is at least about 800,000 subjects. In some embodiments, the reference population for determination of relative IL-33 Asthma PRS score is at least about 900,000 subjects. In some embodiments, the reference population for determination of relative IL-33 Asthma PRS score is at least about 1,000,000 subjects.

In some embodiments, the reference population is not limited in any way to any particular ancestry group. In some embodiments, the reference population is enriched for members of an ancestry group. In some embodiments, the ancestry group is self-reported. In some embodiments, the ancestry group is assigned based upon genetic testing for ancestry. In some embodiments, the ancestry group is derived from a principal component analysis of ancestry. In some embodiments the ancestry group is European. In some embodiments the ancestry group is east European. In some embodiments the ancestry group is west European. In some embodiments the ancestry group is African. In some embodiments the ancestry group is admixed American. In some embodiments the ancestry group is East Asian. In some embodiments the ancestry group is South Asian. In some embodiments the ancestry group is any mixture of any two or more of the European, African, admixed American, East Asian, and South Asian populations.

In some embodiments, the method further comprises initiating a treatment to the subject. The treatment can comprise an ICS, a leukotriene modifier, a long-acting muscarinic antagonist (LAMA), a long-acting beta agonist (LABA), theophylline, combination inhalers that contain both a corticosteroid and a LABA, a short-acting beta agonist such as albuterol, ipratropium, an oral corticosteroid (OCS), an intravenous corticosteroid, an allergy shot, an allergy medication, omalizumab, mepolizumab, benralizumab, reslizumab, dupilumab, and itepekimab, or any combination thereof.

Examples of therapeutic agents useful for treating asthma include, but are not limited to, an ICS, a leukotriene modifier, a long-acting beta agonist (LABA), theophylline, combination inhalers that contain both a corticosteroid and a LABA, a short-acting beta agonist such as albuterol, ipratropium, an oral corticosteroid, an intravenous corticosteroid, an allergy shot, an allergy medication, omalizumab, mepolizumab, benralizumab, reslizumab, dupilumab, and itepekimab, or any combination thereof. A standard amount of any of these agents is determined by a physician in accordance with label guidelines.

Initiating a treatment can include devising a treatment plan based on the risk group, which corresponds to the IL-33 Asthma PRS calculated for the subject. In some embodiments, an IL-33 Asthma PRS is predictive of treatment efficacy or of a subject's response to a therapeutic regimen. Accordingly, the treatment can be determined or adjusted according to the IL-33 Asthma PRS.

In some embodiments, the treatment initiation comprises modifying dosage or regimen of a treatment that a subject with asthma or COPD already receives based on an IL-33 Asthma PRS calculated for the subject. In some embodiments, the treatment initiation comprises substitution of one therapeutic agent with another based on an IL-33 Asthma PRS. In some embodiments, the treatment initiation comprises starting a regimen of a therapeutic agent in addition to a therapeutic agent a subject already receives. In some embodiments, the treatment initiation comprises starting administration of a therapeutic regimen to a previously untreated asthma or COPD subject.

In some embodiments, the therapeutic agent is an IL-33 antagonist, an interleukin-4 receptor alpha antagonist, and/or an interleukin-13 receptor antagonist. In some embodiments, an IL-33 Asthma PRS is predictive of treatment efficacy or of a subject's response to treatment with an IL-33 antagonist, an interleukin-4 receptor alpha antagonist, and/or an interleukin-13 receptor antagonist. Accordingly, the IL-33 antagonist, an interleukin-4 receptor alpha antagonist, and/or an interleukin-13 receptor antagonist treatment can be determined or adjusted according to the IL-33 Asthma PRS calculated for the subject.

As used herein, the term “antagonist” means either that a given compound is capable of inhibiting the activity of the respective protein or other substance in the cell at least to a certain amount. This can be achieved by a direct interaction of the compound with the given protein or substance (“direct inhibition”) or by an interaction of the compound with other proteins or other substances in or outside the cell which leads to an at least partial inhibition of the activity of the protein or substance (“indirect inhibition”). Inhibition of protein activity can also be achieved through suppressing the expression of a target protein. Techniques of inhibiting protein expression include, but not limited to, antisense inhibition, siRNA-mediated inhibition, miRNA mediated inhibition, ribozyme-mediated inhibition, DNA-directed RNA interference (DdRNAi), RNA-directed DNA methylation, transcription activator-like effector nucleases (TALEN)-mediated inhibition, zinc finger nuclease-mediated inhibition, aptamer-mediated inhibition, and CRISPR-mediated inhibition.

As used herein, “antisense inhibition” means reduction of target nucleic acid levels in the presence of an oligonucleotide complementary to a target nucleic acid compared to target nucleic acid levels in the absence of the oligonucleotide.

In some embodiments, the IL-33 antagonist, the interleukin-4 receptor alpha antagonist, and/or the interleukin-13 receptor antagonist is a small molecule.

In some embodiments, the IL-33 antagonist, the interleukin-4 receptor alpha antagonist, and/or the interleukin-13 receptor antagonist is an siRNA.

In some embodiments, the IL-33 antagonist is an anti-IL-33 antibody or an antigen binding portion thereof. In some embodiments, the IL-33 antagonist is an anti-IL-33 receptor antagonist. In some embodiments, the interleukin-4 receptor alpha antagonist is an anti-interleukin-4 receptor alpha antibody or an antigen binding fragment thereof. In some embodiments, the anti-interleukin-4 receptor alpha antagonist is an anti-interleukin-4 antibody or an antigen binding fragment thereof. In some embodiments, the interleukin-13 receptor antagonist is an anti-IL-13 receptor antibody. In some embodiments, the interleukin-13 receptor antagonist is an anti-interleukin-13 antibody or antigen-binding fragment thereof.

The term “antibody,” as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-33 antibody (or antigen-binding fragment thereof) or the anti-interleukin-4 receptor alpha (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Interleukin-4 receptor alpha antagonists include, but are not limited to, dupilumab and pitrakinra. Interleukin-13 receptor antagonists include, but are not limited to, dupilumab, tralokinumab, pitrakinra, and lebrikizumab. A standard dosage amount of dupilumab for adults and adolescents (12 years of age and older) is: i) an initial dose of 400 mg (two 200 mg injections) followed by 200 mg administered every other week; or ii) an initial dose of 600 mg (two 300 mg injections) followed by 300 mg administered every other week; or iii) for patients requiring concomitant oral corticosteroids or with co-morbid moderate-to-severe atopic dermatitis for which dupilumab is indicated, starting with an initial dose of 600 mg followed by 300 mg administered every other week. In some embodiments, the interleukin-4 receptor alpha antagonist is not an interleukin-13 receptor antagonist. In some embodiments, the interleukin-13 receptor antagonist is not an IL4R alpha antagonist. In some embodiments, the interleukin-4 receptor alpha antagonist and/or the interleukin-13 receptor antagonist are separate antagonists. Where the interleukin-4 receptor alpha antagonist and/or the interleukin-13 receptor antagonist are separate antagonists, a first separate antagonist is an interleukin-4 receptor alpha antagonist but not an interleukin-13 receptor antagonist and a second separate antagonist is an interleukin-13 receptor antagonist but not an interleukin-4 receptor alpha antagonist, i.e., two separate antagonists are administered.

In some embodiments, the interleukin-4 receptor alpha antagonist specifically binds to human IL-4Rα and comprises a heavy chain variable region (HCVR) comprising SEQ ID NO:1 and a light chain variable region (LCVR) comprising SEQ ID NO:2, a heavy chain complementarity determining region 1 (HCDR1) comprising SEQ ID NO:3, a HCDR2 comprising SEQ ID NO:4, a HCDR3 comprising SEQ ID NO:5, a light chain complementarity determining region 1 (LCDR1) comprising SEQ ID NO:6, a LCDR2 comprising SEQ ID NO:7, and a LCDR3 comprising SEQ ID NO:8. The full-length heavy chain of dupilumab is shown as SEQ ID NO:9 and the full length light chain is shown as SEQ ID NO:10. Human anti-IL-4R antibodies can be generated as described in U.S. Pat. No. 7,608,693.

In some embodiments, the IL-33 antagonist comprises itepekimab. In some embodiments, the interleukin-4 receptor alpha antagonist is dupilumab. In some embodiments, the interleukin-13 receptor antagonist is dupilumab. In some embodiments, the interleukin-4 receptor alpha antagonist and the interleukin-13 receptor antagonist is dupilumab.

In the context of the methods disclosed herein, additional therapeutically active component(s), e.g., any of the agents listed above or derivatives thereof, may be administered just prior to, concurrent with, or shortly after the administration of an IL-33 antagonist, an interleukin-4 receptor alpha antagonist, and/or an interleukin-13 receptor antagonist; (for purposes of the present disclosure, such administration regimens are considered the administration of an IL-33 antagonist, an interleukin-4 receptor alpha antagonist, and/or an interleukin-13 receptor antagonist “in combination with” an additional therapeutically active component). In some embodiments, an additional therapeutically active component is considered administered “in combination with” an IL-33 antagonist, an interleukin-4 receptor alpha antagonist, and/or an interleukin-13 receptor antagonist notwithstanding the fact that the additional therapeutically active component and the IL-33 antagonist, the interleukin-4 receptor alpha antagonist, and/or the interleukin-13 receptor antagonist are administered by different routes. The present methods include pharmaceutical compositions and methods of use thereof in which an IL-33 antagonist, an interleukin-4 receptor alpha antagonist, and/or an interleukin-13 receptor antagonist is co-formulated with one or more of the additional therapeutically active component(s) as described herein.

As used herein, the terms “treat”, “treatment”, or “treating” refers to administering a therapeutic agent for prophylactic and/or therapeutic purposes.

Examples of therapeutic agents that are useful for treating a lung disease include, but are not limited to, an ICS, a leukotriene modifier, a long-acting beta agonist (LABA), theophylline, combination inhalers that contain both a corticosteroid and a LABA, a short-acting beta agonist such as albuterol, ipratropium, an oral corticosteroid, an intravenous corticosteroid, an allergy shot, an allergy medication, omalizumab, mepolizumab, benralizumab, reslizumab, dupilumab, and itepekimab, or any combination thereof.

As used herein, the term “therapeutic treatment” refers to administering a therapeutic agent to a subject to a subject having a lung disease.

As used herein, the terms “prophylactic treatment” or “prophylaxis” refers to administration of a subject who is not currently nor ever has had at least one lung disease.

As used herein, the term “a lung disease” refers to, without limitation, asthma, COPD, chronic bronchitis, emphysema, acute bronchitis, cystic fibrosis, a bacterial lung infection, a mycobacterial infection, pneumonia, tuberculosis caused by, without limitation, Mycobacterium tuberculosis, pulmonary edema, lung cancer, acute respiratory distress syndrome (ARDS) including, without limitation, ARDS related to COVID, pneumoconiosis, lung damage caused by a chemical, biological, or radio-nuclear (CBRN) agent, black lung disease relating from exposure to coal dust, and asbestosis relating to exposure to asbestos.

In some embodiments, the asthma can be, without limitation, mild asthma, moderate asthma, severe asthma, eosinophilic asthma with or without changes to immunoglobulin E levels, or oral corticosteroid-dependent asthma.

In some embodiments, asthma exacerbation can be annualized asthma exacerbation.

Examples of compounds for treating acute asthma exacerbation include, but are not limited to oral corticosteroids or an increase in the dose of inhaled corticosteroids. Examples of compounds for preventing acute asthma exacerbation include, but are not limited to inhaled corticosteroids (including combination with long-acting beta agonists), oral corticosteroids, dupilumab, mepolizumab, benralizumab, reslizumab, omalizumab, tezepelumab, and azithromycin.

Examples of compounds for treating a loss of asthma control include, but are not limited to any available asthma therapy such as, for example, an ICS, a leukotriene modifier, a LABA, a LAMA, theophylline, combination inhalers that contain both a corticosteroid and a LABA, a short-acting beta agonist such as albuterol, ipratropium, an OCS, an intravenous corticosteroid, an allergy shot, an allergy medication, omalizumab, mepolizumab, benralizumab, reslizumab, dupilumab, and itepekimab, or any combination thereof.

Examples of compounds for decreasing an eosinophil count include, but are not limited to OCS, mepolizumab, benralizumab, reslizumab, omalizumab, and tezepelumab, or any combination thereof.

Examples of compounds for increasing an FEV1 value include, but are not limited to OCS, ICS, LABA, LAMA, a short-acting muscarinic antagonist (SAMA), a short-acting beta agonist (SABA), an anti-leukotriene (such as montelukast), theophylline, dupilumab, tezepelumab, omalizumab, mepolizumab, benralizumab, and reslizumab, or any combination thereof.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Generation of Polygenic Risk Scores

### Datasets:

A pathway-specific asthma PRS for a European population was generated. The PRS consisted of 543 variants near or in the IL-33 and IL1RL1 genes. Individual SNP contribution was estimated from an asthma GWAS using the LDpred method. Additionally, a pathway-specific asthma PRS for an admixed American population was generated. This comprised 205 variants near or in the IL-33 and IL1RL1 genes. Individual SNP contribution was estimated from an asthma GWAS using the LDpred method.

### PRS Algorithm Selection:

The LDpred approach to generating polygenic risk scores was used. LDpred is a Bayesian approach to PRS development that calculates a posterior mean effect (adjusted effect size) for all variants based on a prior and LD information from a reference panel. Heuristically, the effect sizes generated from LDpred differs from P&T in that LDpred jointly models the effect size and variance of each marker, incorporating the LD structure when shrinking the effect sizes. Adjustment or shrinkage of variant weights is based not only on magnitude of variant association with disease but also linkage disequilibrium (LD) between variants. For the LDpred approach, 1000 Genomes phase 3 version 5 data was used for the LD reference panel.

### Prs Calculation:

From the LDpred approach, a set of variants and their respective weights were generated. In the case of LDpred, the variant weights were the adjusted log odds ratio (posterior mean). After generation of weights, the process for calculating and normalizing scores was identical. For a set of i=1, . . . ,M variants in j=1, . . . N patients, the PRS for patient j was calculated by: PRSᵢⱼ=Σᵢ₌₁^{M}(Bᵢxᵢⱼ), where Bᵢ is the log odds ratio for variant i and xᵢⱼ is the number of risk alleles carried by patient j at variant i (for imputed variants, the allele dosage for variant i). Scores were standardized to ^{˜}N(0,1) by subtracting the mean PRS and dividing by the PRS standard deviation within each ancestry group.

### Testing and Validating PRS Algorithms:

For each set of LDpred tuning parameter, a PRS was calculated and a logistic regression run with the composite endpoint as the dependent variable and PRS, age, sex, and ancestry covariates as independent variables. The odds ratio (OR) per PRS standard deviation (SD) and area under the curve (AUC) were reported for each model.

### Selection of a Threshold for Defining High Risk:

Genetic high risk was defined as patients within the 25th, 50th, 75th, and 100th percentile of the distribution of the polygenic risk scores. This threshold was selected in a post hoc analysis, which evaluated high genetic risk thresholds ranging from the 25th, 50th, 75th, and 100th percentile.

### Statistical Analysis:

Baseline disease and medical history characteristics were analyzed to assess the distribution of asthma risk factors by genetic risk status, high (>percentile threshold) vs lower percentile threshold). Continuous baseline characteristics were compared using a t-test, and binary or categorical characteristics were tested with a chi-square or Fisher's exact test.

### Results:

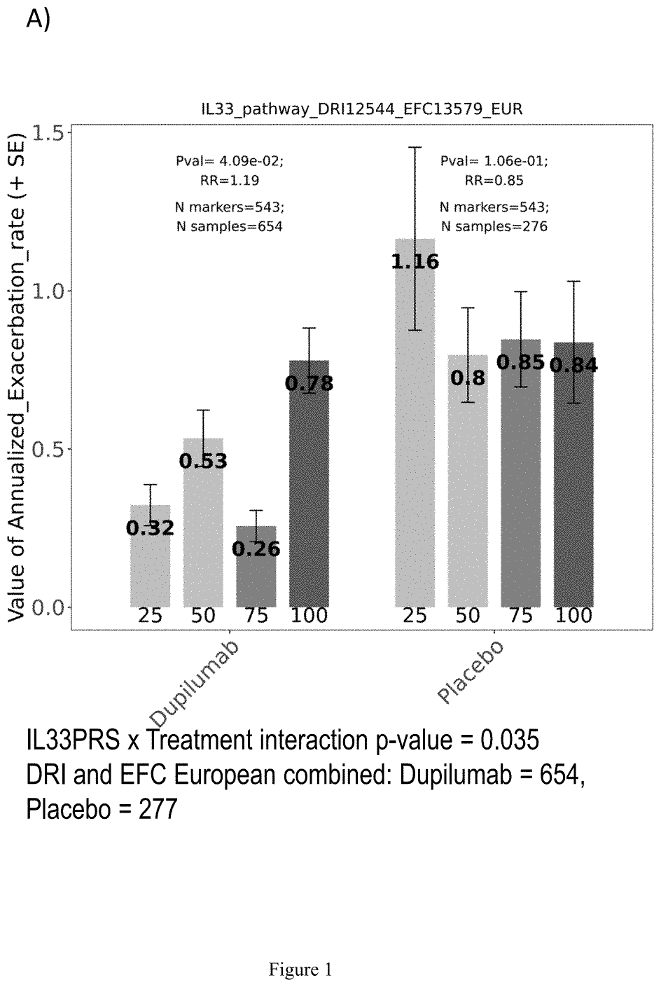

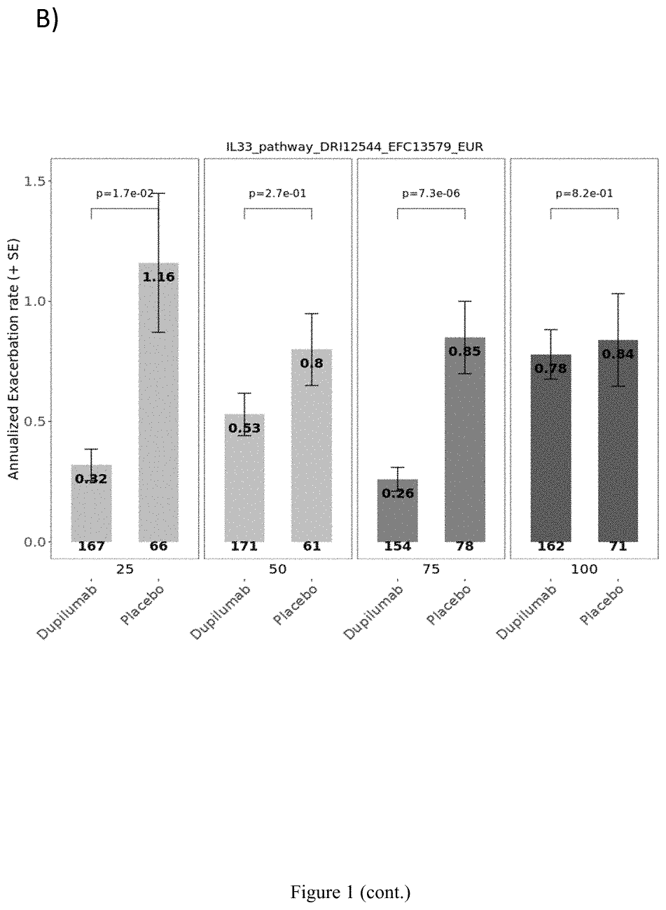

IL-33 Asthma PRS determined using the LDpred method is significantly associated with higher annualized exacerbation rate in dupilumab-treated European patients. Both graphs of FIG. 1 show an association of IL-33 Asthma PRS and annualized exacerbation rates. In particular, Panel A shows genetic effect for patients receiving the same treatment. The X-axis is the IL33 PRS score divided into quartiles ranked from lowest to the highest, and the Y-axis is annualized exacerbation rate. In dupilumab treated arm, those with higher IL33 PRS showed higher exacerbation rate. p value is 0.41. It is also significant when comparing those with the top quartile to the rest or the lowest quartile, suggesting people at highest IL33 PRS quartile are not getting as much benefit as those at lower IL33 PRS quartile ones. In particular, Panel B shows a comparison of treatment effect conditional on IL33 PRS quartiles. Among lower IL33 PRS quartiles, dupilumab treated patients had significant lower exacerbation rate. At highest IL33 PRS quartile, no differences was seen between dupilumab vs. placebo arm.

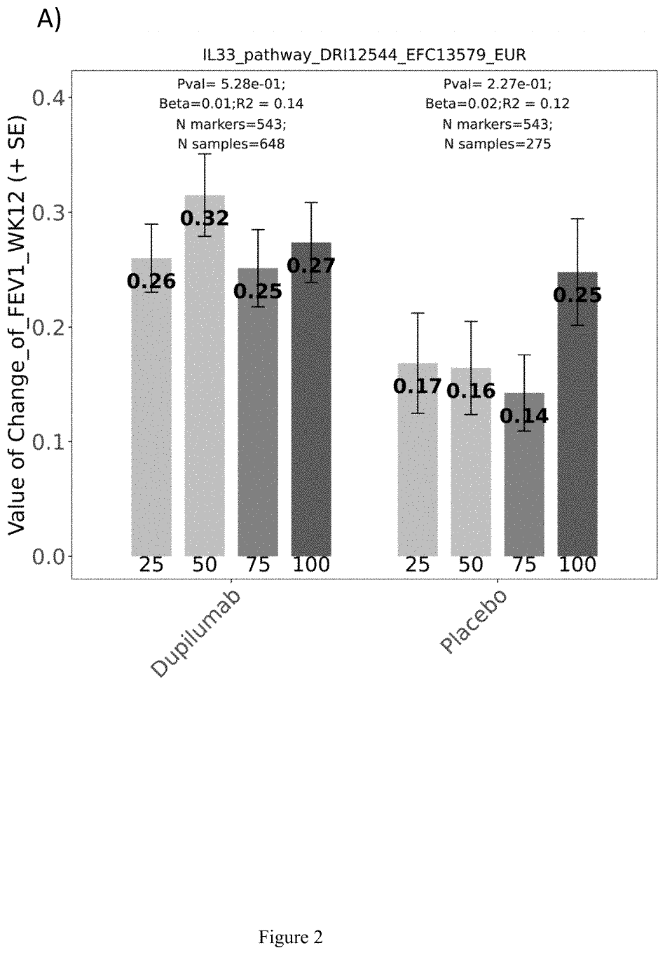

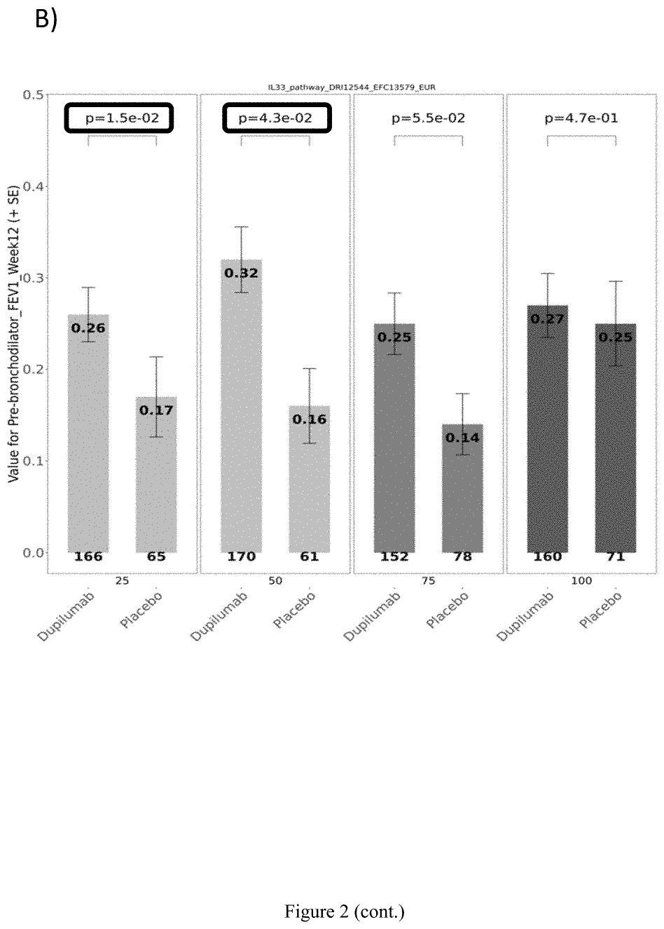

IL-33 Asthma PRS determined using the LDpred is not associated with a change of pre-bronchodilator FEV1 at week 12 in dupilumab-treated European patients, however when compared to placebo treated patients, patients with the lower IL-33 Asthma PRS scores had the greatest clinical benefit. In the study disclosed herein, the other co-primary endpoint was the change of FEV1 change (FIG. 2). In particular, Panel A shows that when comparing the treatment effect, dupilumab treatment significantly increased FEV1 value for the patients with lower IL33 PRS quartiles, but not for those at higher PRS quartiles.

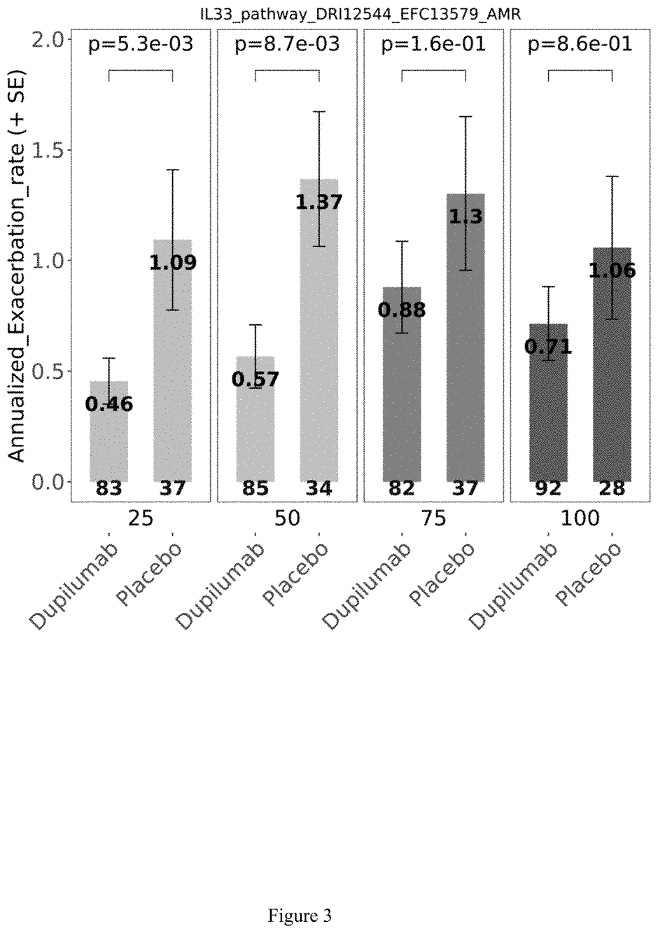

IL-33 Asthma PRS determined using the LDpred method was trending to be associated with higher annualized exacerbation rate in dupilumab-treated admixed American patients. FIG. 3 shows that when comparing dupilumab treatment and placebo arms, the higher IL33 PRS quartile subjects (75 and 100 groups) are significantly associated with less response efficacy to dupilumab.

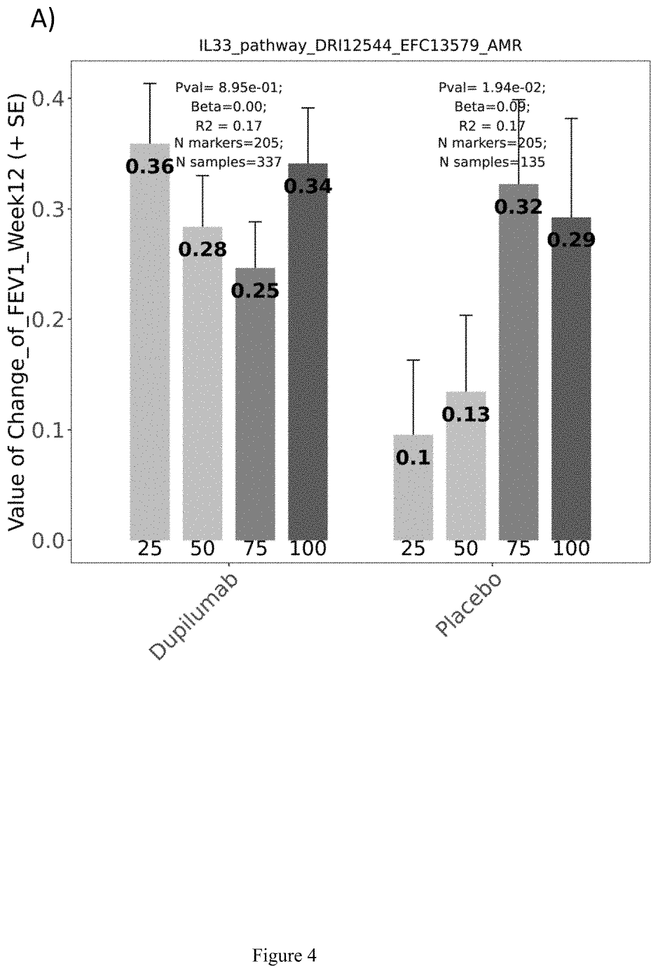

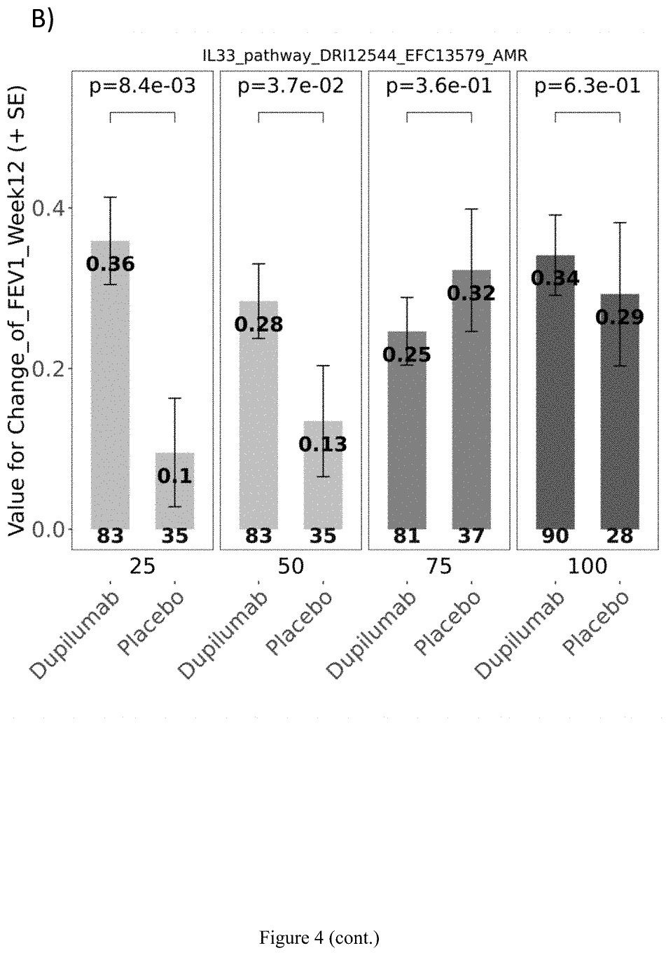

IL-33 Asthma PRS determined using the LDpred method was not associated with a change of FEV1 at week 12 in dupilumab-treated admixed American patients, however when compared to placebo treated patients, patients with the lower IL-33 Asthma PRS scores had the greatest clinical benefit. FIG. 4 shows a sample-wise distribution of variants. For the graph in Panel A, the IL-33 Asthma PRS divided into quartiles and ranked from lowest to the highest and the number inside each bar is the change in FEV1 at week 12. For the graph in Panel B, the IL-33 Asthma PRS divided into quartiles and ranked from lowest to the highest, the number at the bottom of each bar is the number of patients in each group, and the change in FEV1 at week 12.

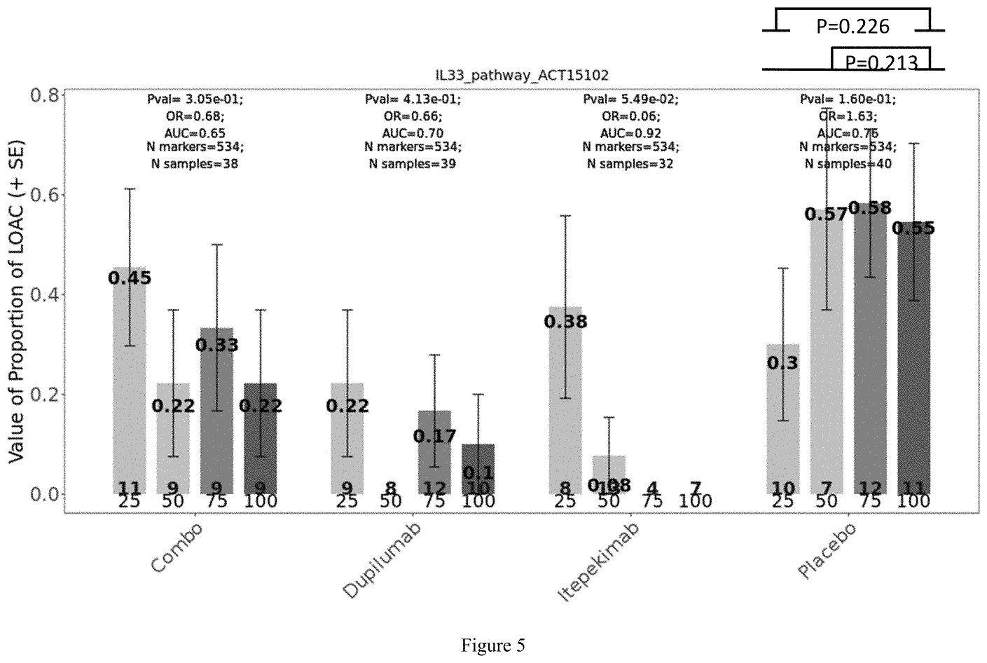

IL-33 Asthma PRS determined using the LDpred method showed a trend for decreased LOAC risk in itepekimab-monotreated European subjects. With 148 European samples, the IL33 asthma PRS for European patients was applied (FIG. 5). The IL-33 Asthma PRS divided into quartiles and ranked from lowest to the highest, the number at the bottom of each bar is the number of subjects in each group, and the number inside each bar is the proportion of subjects with LOAC. It was found that the IL-33 Asthma PRS trended in association with decreased LOAC (p value=0.055). Comparing the lowest quartile to the other quartiles, it was observed that the higher IL-33 Asthma PRS patients had less LOAC compared to the lowest quartile. The variation of the IL-33 Asthma PRS with treatment regimen was not significant. However, this may be due to the small sample size of this study. FIG. 5 shows a trend in itepekimab monotherapy arm where patients with higher IL33 PRS score showed less LOAC events.

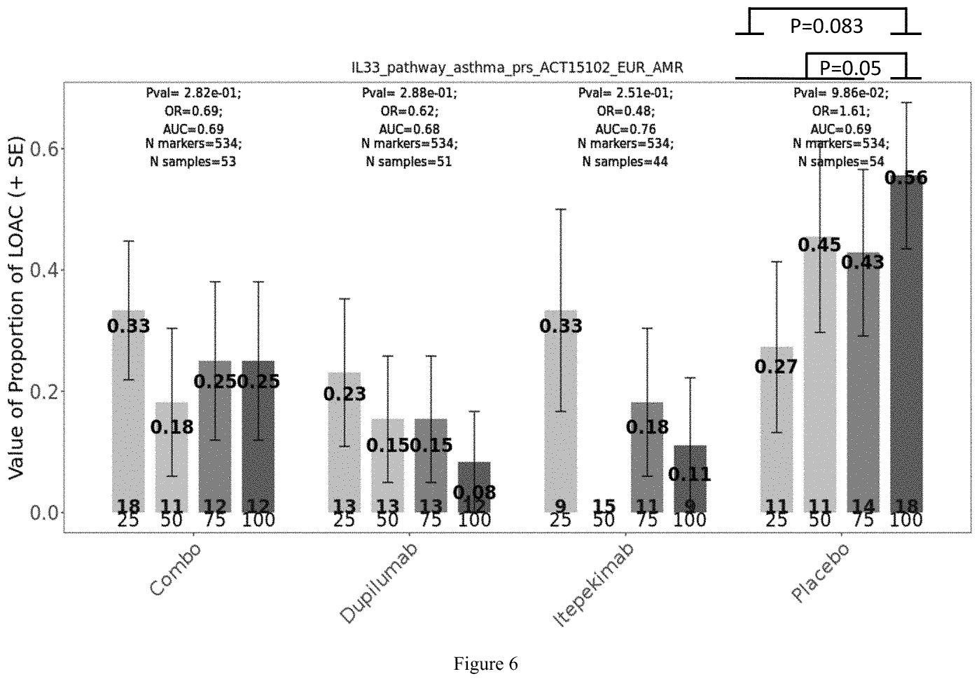

IL-33 Asthma PRS showed a trend for decreased LOAC risk in itepekimab-monotreated European and admixed American subjects. The IL-33 Asthma PRS for the remaining 50 admixed American samples in the itepekimab trial were applied and combined with those of European subjects (FIG. 6). The IL-33 Asthma PRS divided into quartiles and ranked from lowest to the highest, the number at the bottom of each bar is the number of subjects in each group, and the number inside each bar is the proportion of subjects with LOAC. Again, the asthma patients with higher IL-33-Asthma PRS had less LOAC than the patients in the lowest quartile for the itepekimab monotherapy arm. This finding suggested that asthma patients who have a higher IL-33 Asthma PRS may benefit more from itepekimab treatment. In the placebo arm, the IL-33 Asthma PRS trended to being associated with increased LOAC. This suggested that the placebo asthma patients with a higher IL33 Asthma PRS suffer from more LOAC. FIG. 6 shows that in the itepekimab monotherapy arm, the asthma patients with higher IL33 PRS had less LOAC than the ones in the lowest quartile, suggesting asthma patients who have a higher IL33-based genetic risk, may benefit more from the itepekimab treatment. In the placebo arm, the IL33 PRS has a trending being associated with increased LOAC, suggesting the asthma patients in higher IL33 PRS quartile suffer from more LOAC.

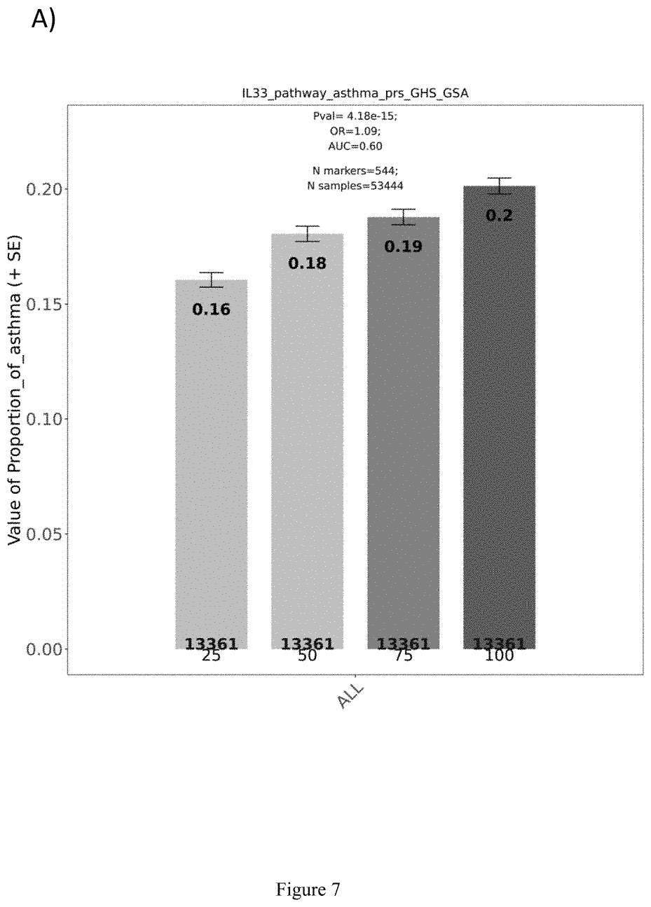

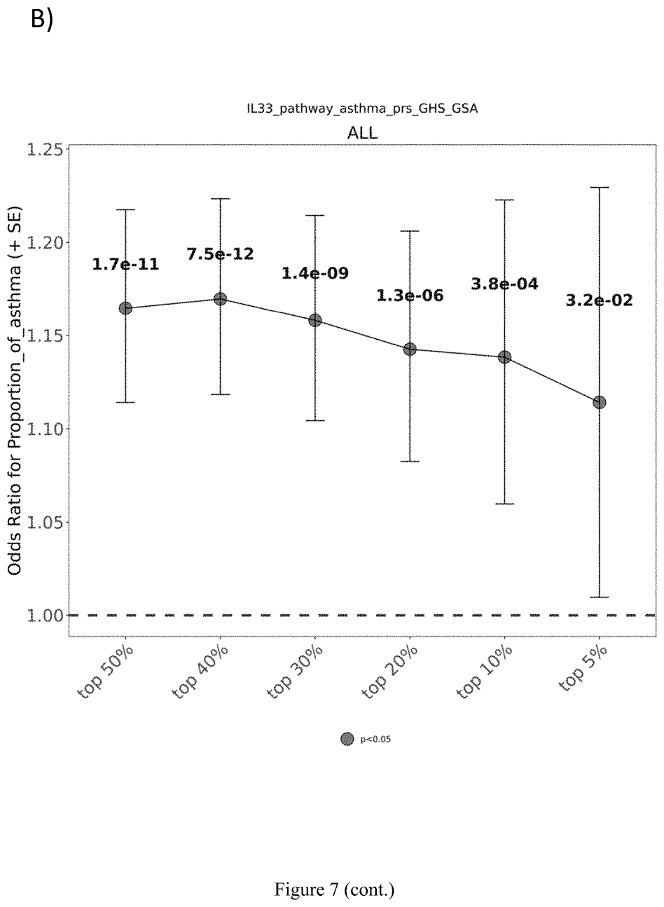

IL-33 Asthma PRS determined using the LDpred method was significantly associated with asthma in GHS_GSA European subjects. The GHS_GSA population is a cohort in the Geisinger Health System. The IL-33 asthma PRS was strongly associated with asthma risk (p value=4.18×10⁻³⁰) (FIG. 7). For the graph in Panel A, the IL-33 Asthma PRS divided into quartiles and ranked from lowest to the highest, the number at the bottom of each bar is the number of subjects in each group, and the number inside each bar is the proportion of subjects with asthma. For the graph in Panel B, the IL-33 Asthma PRS percentiles and the ordinate is the odds ratio for the proportion of subjects with asthma. FIG. 7 shows validated IL33 asthma PRS in correlation with asthma risk. IL33 Asthma PRS determined by LDpred was significantly associated with Asthma in GUS-GSA European Subjects.

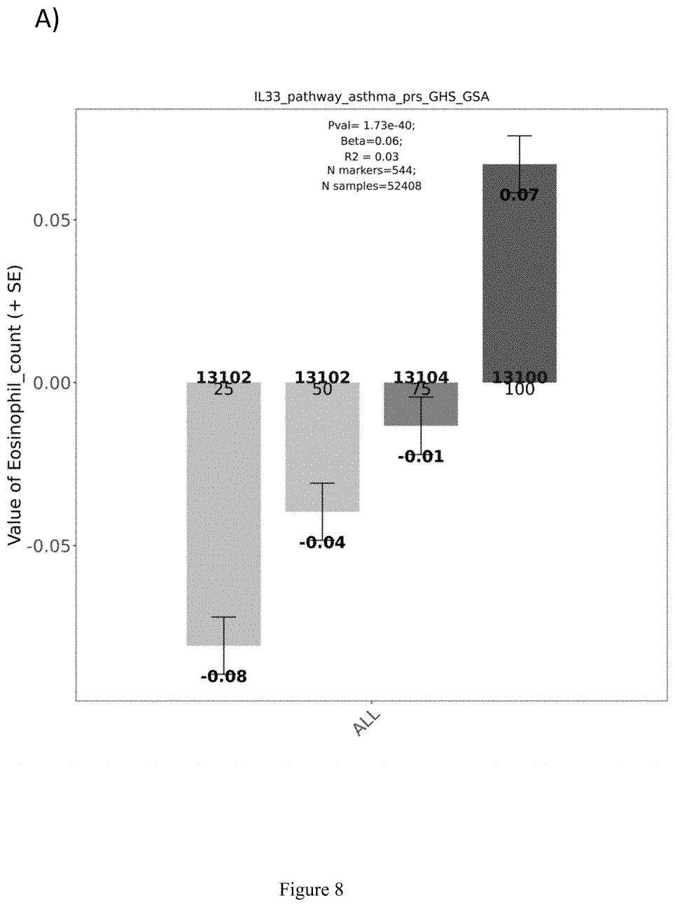

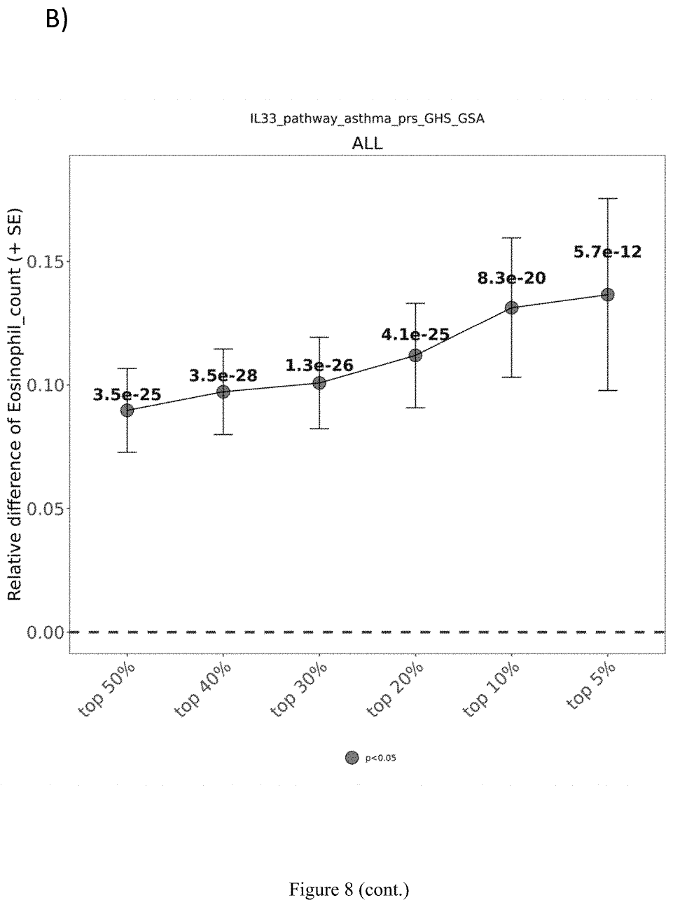

IL-33 Asthma PRS determined using the LDpred method was significantly associated with eosinophil count in GHS_GSA European subjects. The IL-33 Asthma PRS determined using the LDpred method also showed a significant association with eosinophil count (p value=1.73×10⁻⁴⁰) (FIG. 8). For the graph in Panel A, the numbers “25,” “50,”75,” and “100” are the IL-33 Asthma PRS divided into quartiles and ranked from lowest to the highest, the numbers “13102, 13102,” “13104,” and “13101” are the number of subjects in each group, and the number inside each bar is the eosinophil count. For the graph in Panel B, the IL-33 Asthma PRS percentiles and the ordinate is the relative difference of the eosinophil count. FIG. 8 shows that the IL33 PRS from Ldpred method also shows significant positive correlation with eosinophil count.

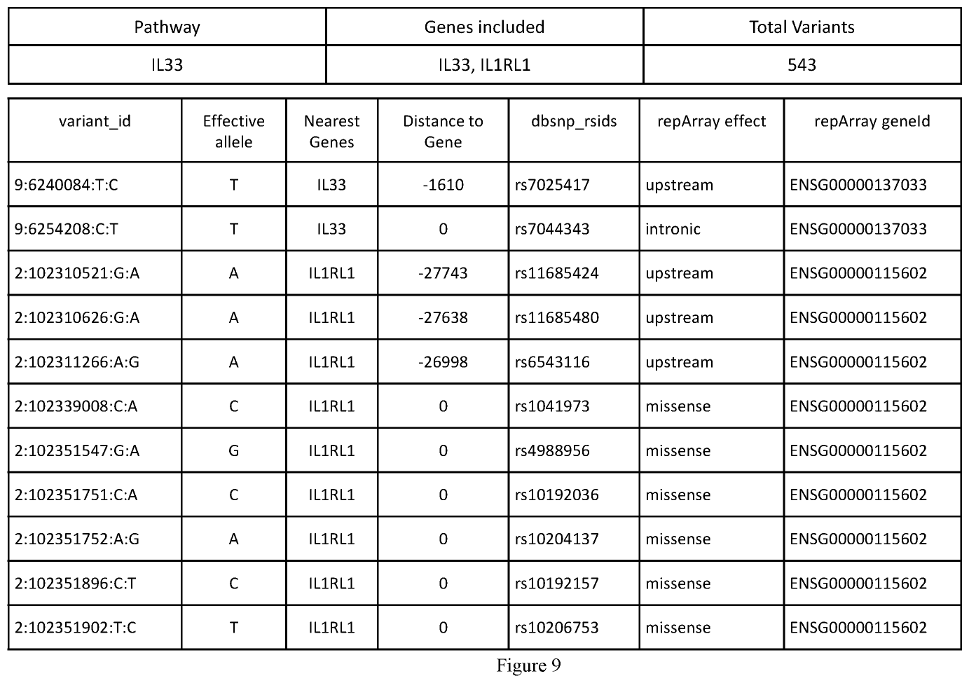

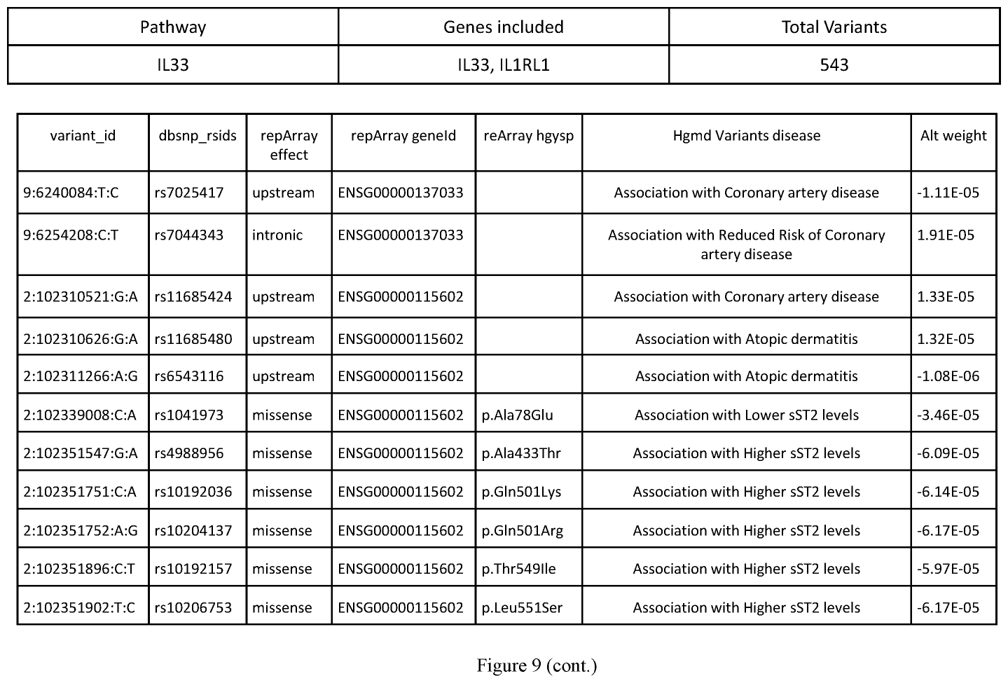

11 out of 543 variants in an IL-33 Asthma PRS determined with LDpred training were disease- or trait-associated variants in the HGMD. Variants from the IL-33 Asthma PRSs and interleukin-1 receptor ligands are found in the HGMD database and are associated with disease traits (FIG. 9). The results of the analysis of the 543 variants is set forth in FIG. 9. IL-33 Asthma PRSs and interleukin-1 receptor ligands were generated.

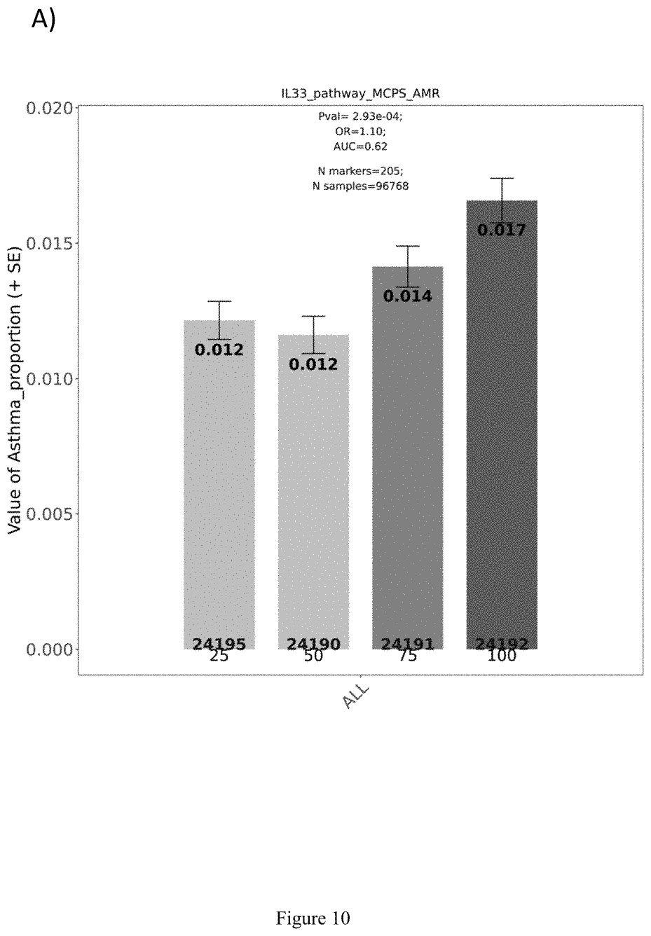

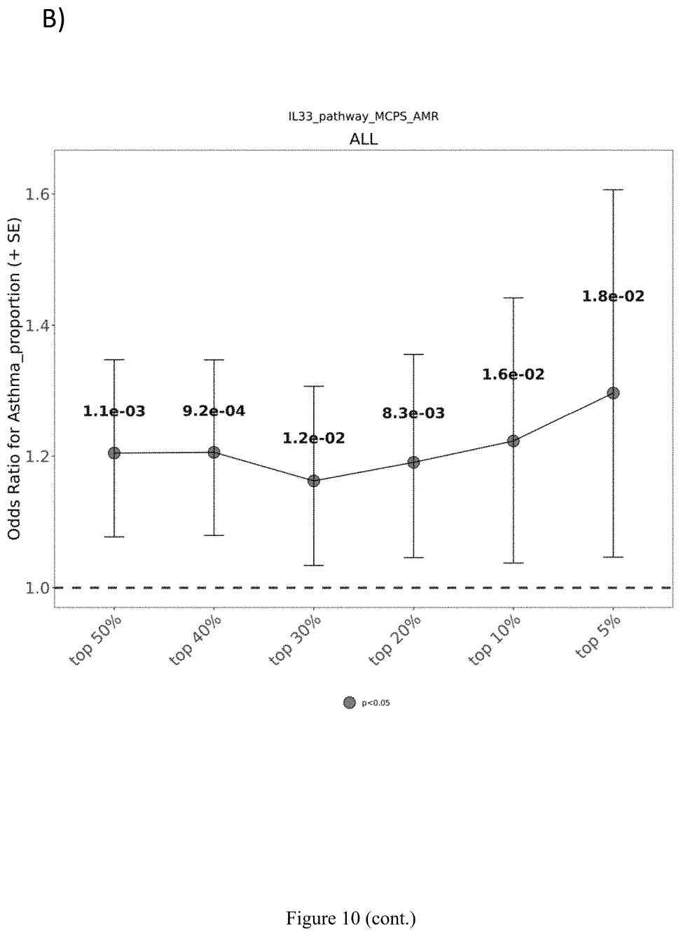

IL-33 asthma polygenic risk score for admixed American subjects (IL-33 AMR Asthma PRS) determined using the LDpred method was significantly associated with asthma risk in Mexico City admixed American subjects. The graphs set forth in FIG. 10 show how the IL-33 asthma PRS for admixed American patients predicts asthma risk in Mexico City admixed American patients. For the graph in Panel A, the IL-33 Asthma PRS divided into quartiles and ranked from lowest to the highest, the number at the bottom of each bar is the number of subjects in each group, and the number inside each bar is the proportion of subjects with asthma. For the graph in Panel B, the IL-33 Asthma PRS percentiles and the ordinate is the odds ratio for the proportion of subjects with asthma. A highly significant association was noted. This finding was applied to the IL-33 Asthma PRSs on clinical samples and combined for both European and admixed American samples together to increase the power. The plot in FIG. 10 shows how IL33 AMR asthma PRS works in predicament asthma risk in MCPS, where there is a highly significant association. This IL33 AMR PRS can be applied on clinical samples and combined both EUR and AMR samples together to increase the power.

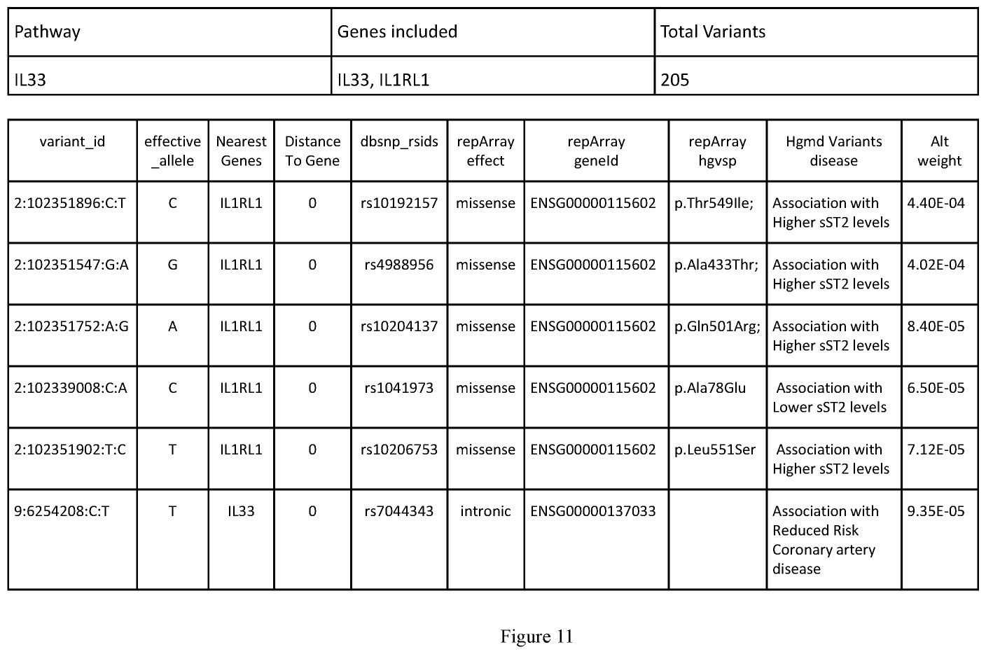

6 out of 205 variants in an IL-33 AMR Asthma PRS determined with LDpred training were associated with diseases or traits in the HGMD. Variants from the AMR IL-33 Asthma PRSs and interleukin-1 receptor ligands are found in the HGMD database and are associated with disease traits (FIG. 11). The results of the analysis of the 543 variants are set forth in FIG. 9. IL-33 Asthma PRSs and interleukin-1 receptor ligands were generated. FIG. 11 shows IL33 PRS and IL4R PRS on clinical samples, and that IL4R PRS showed no significant association for efficacy endpoints.

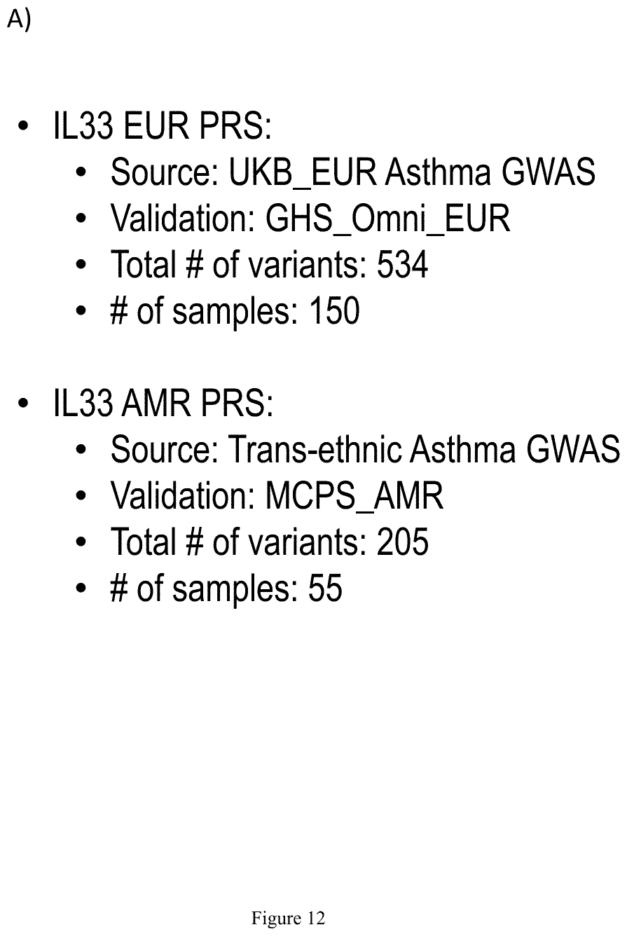

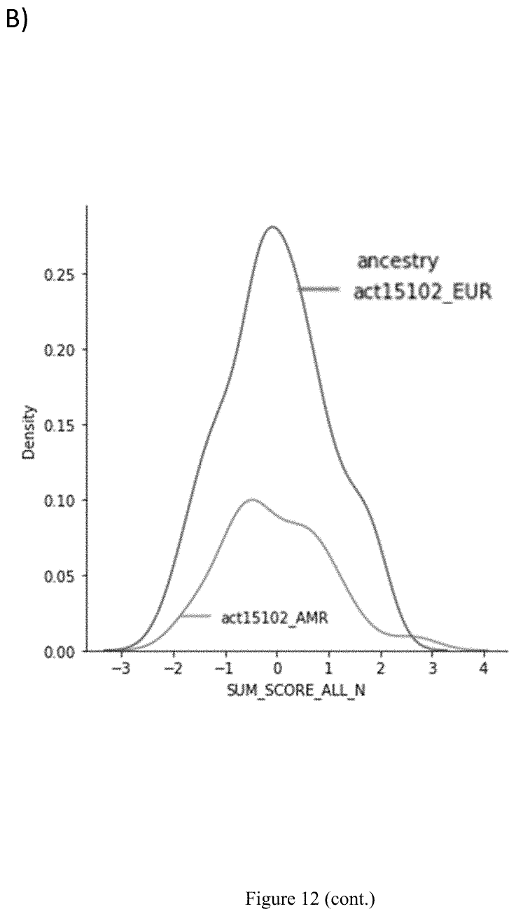

An IL-33 Asthma PRS was determined with LDpred training in a trial of itepekimab-treated European and admixed American combined subjects. FIG. 12 sets forth information on the number of variants and datasets used for generation of the PRS (Panel A) and a sample-wide distribution of PRS scores for European and admixed American subsets (Panel B).

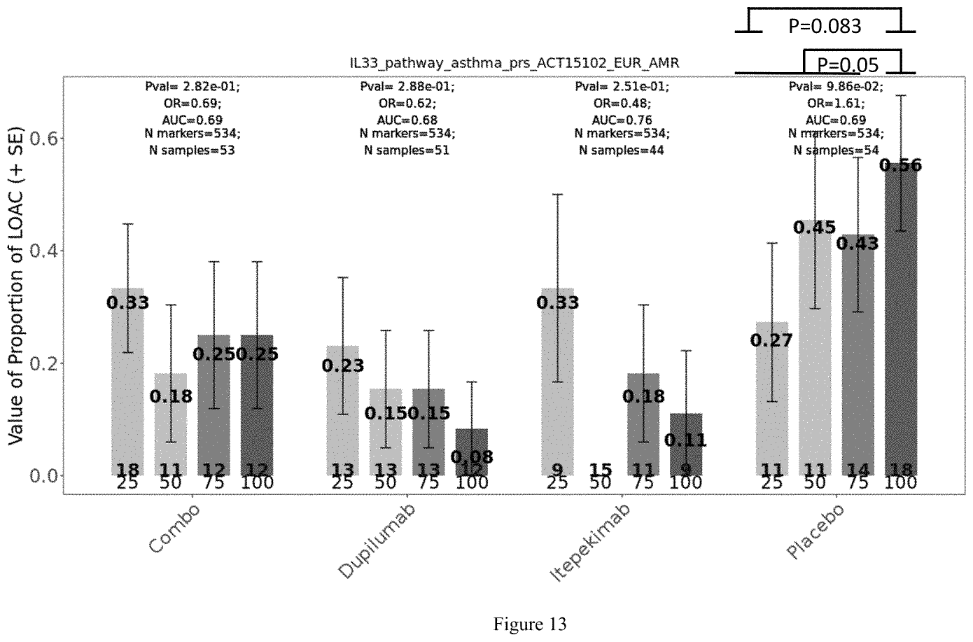

IL-33 Asthma PRS trended for decreased LOAC Risk in itepekimab-monotreated European and admixed American subjects. IL-33 Asthma PRSs for admixed American subjects were applied to the 50 admixed American samples for the itepekimab trial and combined with those of European subjects (FIG. 13). The IL-33 Asthma PRS divided into quartiles and ranked from lowest to the highest, the number at the bottom of each bar is the number of subjects in each group, and the number inside each bar is the proportion of subjects with LOAC. Again, it was observed in the itepekimab monotherapy arm that asthma patients with higher IL-33 Asthma PRSs had less LOAC than the patients in the lowest quartile. This finding suggests that asthma patients who have a higher IL-33-based genetic risk may benefit more from itepekimab treatment. In the placebo arm, the IL-33 Asthma PRS was observed to trend with increased LOAC. This finding suggests that the asthma patients having a higher IL-33 PRS suffer from more LOAC.

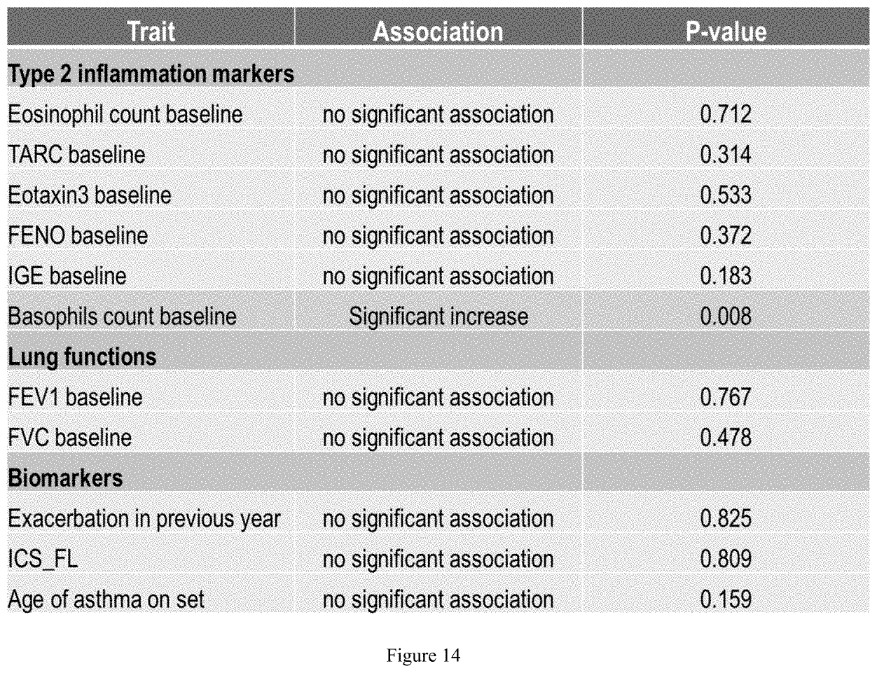

IL-33 Asthma PRS was associated with an increased baseline basophil count for European subjects in trials designated DR112544 and EFC13579. This finding was significant with a p value of 0.008 (FIG. 14).

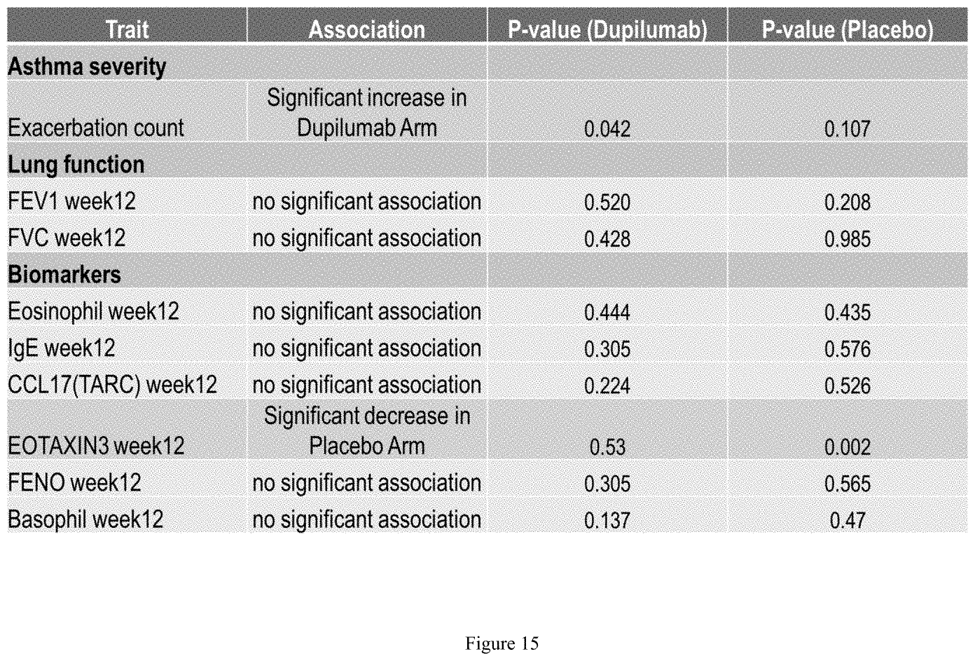

IL-33 Asthma PRS was significantly associated with increased asthma exacerbation in dupilumab-treated European subjects in trials designated DR112544 and EFC13579. A high IL-33 Asthma PRS was associated with a significant increase in asthma exacerbation in dupilumab-treated patients (FIG. 15). The IL-33 Asthma PRS was also associated with a decrease in change of exotoxin-3 levels in the placebo arms.

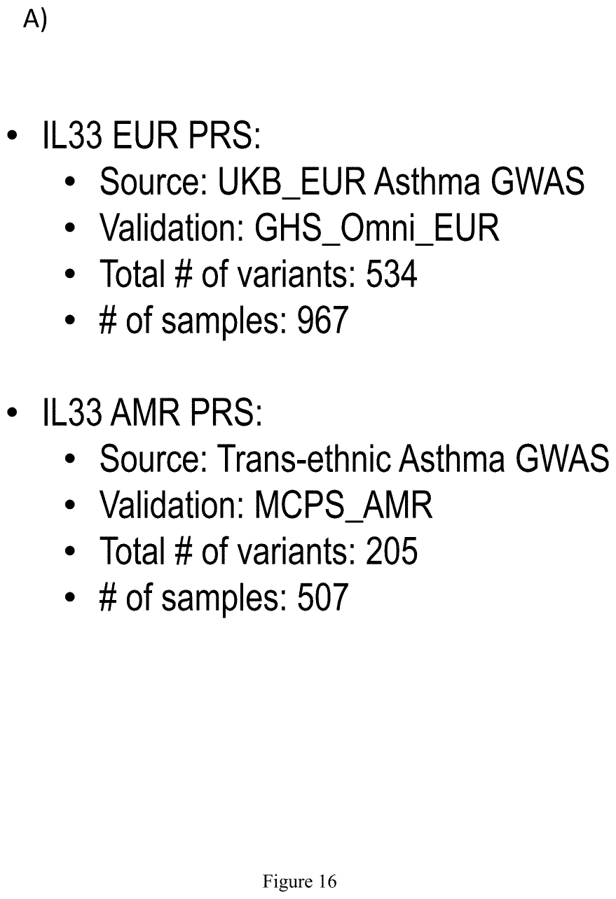

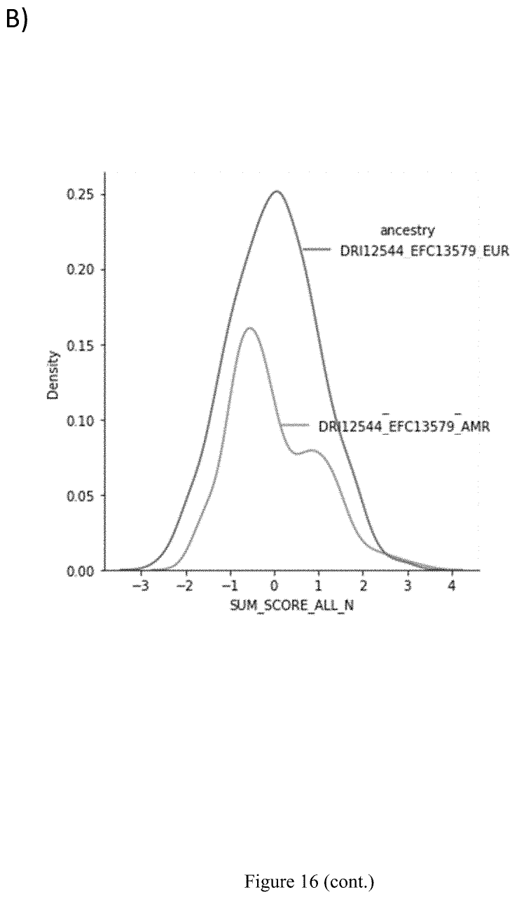

A combined IL-33 Asthma PRS determined with LDpred training for European and admixed American subjects in trials designated DR112544 and EFC13579. FIG. 16 shows information on number of variants and datasets used for generation of the PRS (Panel A) and a sample-wide distribution PRS scores for European and admixed American subjects (Panel B).

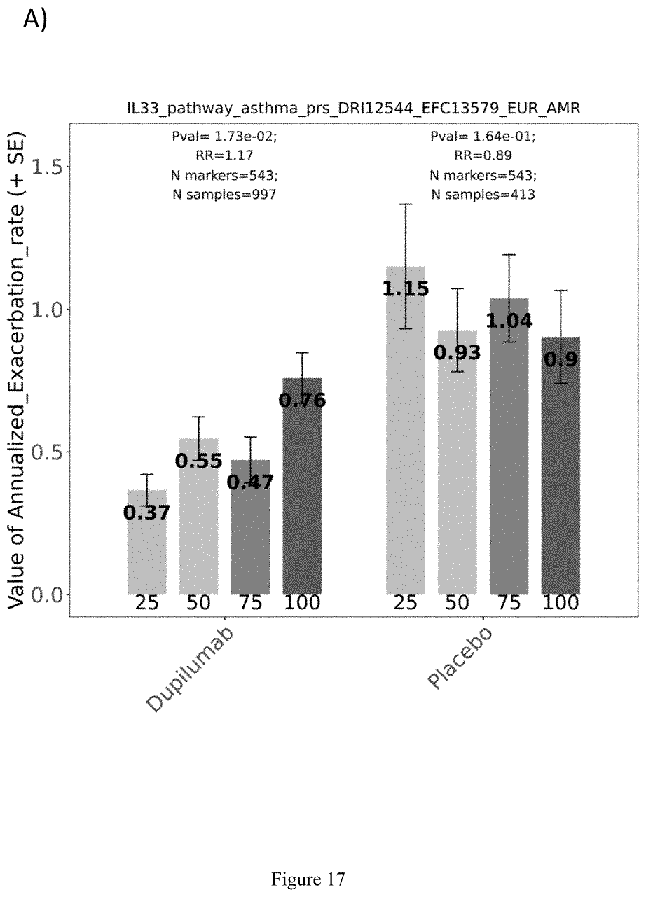

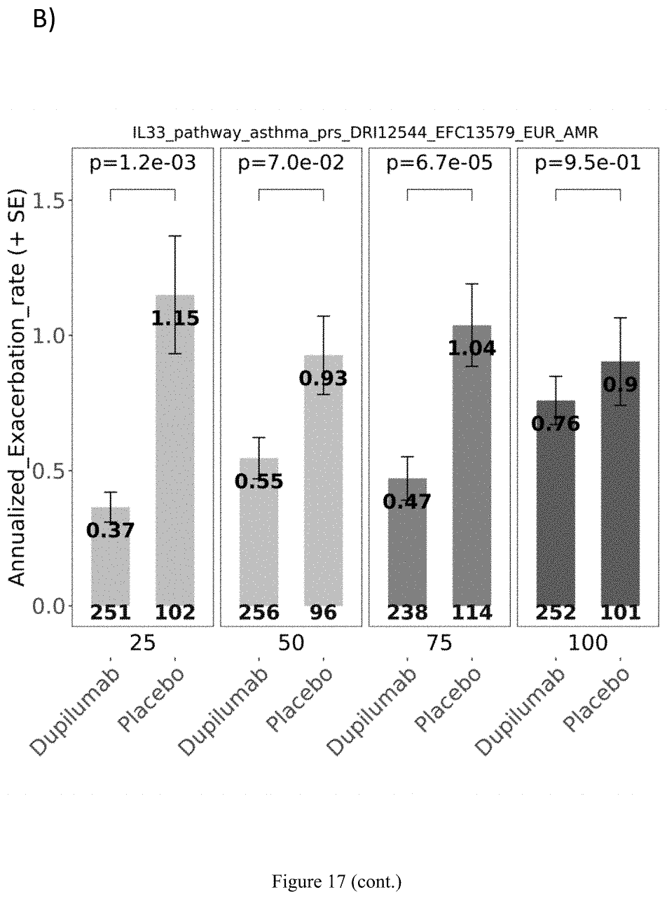

IL-33 Asthma PRS was significantly associated with an increased asthma exacerbation rate in dupilumab-treated European and admixed American combined subjects in trials designated DR112544 and EFC13579. European and admixed American samples were scored separately for their respective IL-33 Asthma PRS. The scores were then combined and regression against the asthma exacerbation rate (FIG. 17). For the graph in Panel A, the IL-33 Asthma PRS divided into quartiles and ranked from lowest to the highest and the number inside each bar is the annualized exacerbation rate. For the graph in Panel B, the IL-33 Asthma PRS divided into quartiles and ranked from lowest to the highest, the number at the bottom of each bar is the number of subjects in each group, and the ordinate is the annualized exacerbation rate. These findings were replicated in European subjects where the IL-33 Asthma PRS was significantly associated with increased exacerbation in the dupilumab arm. Moreover, conditioned on IL-33 Asthma PRS quartiles, the effect associated with dupilumab was significant in the 25th, 50th, and 75th quartile, but not the highest quartile. This suggests that dupilumab's benefits mainly stem from asthma patients with low IL33 Asthma PRSs for dupilumab. FIG. 17 shows that patients having IL-33 Asthma PRS score in the top 25% responded to dupilumab with less efficacy, compared to patients having lower IL-33 Asthma PRS scores and treated with dupilumab.

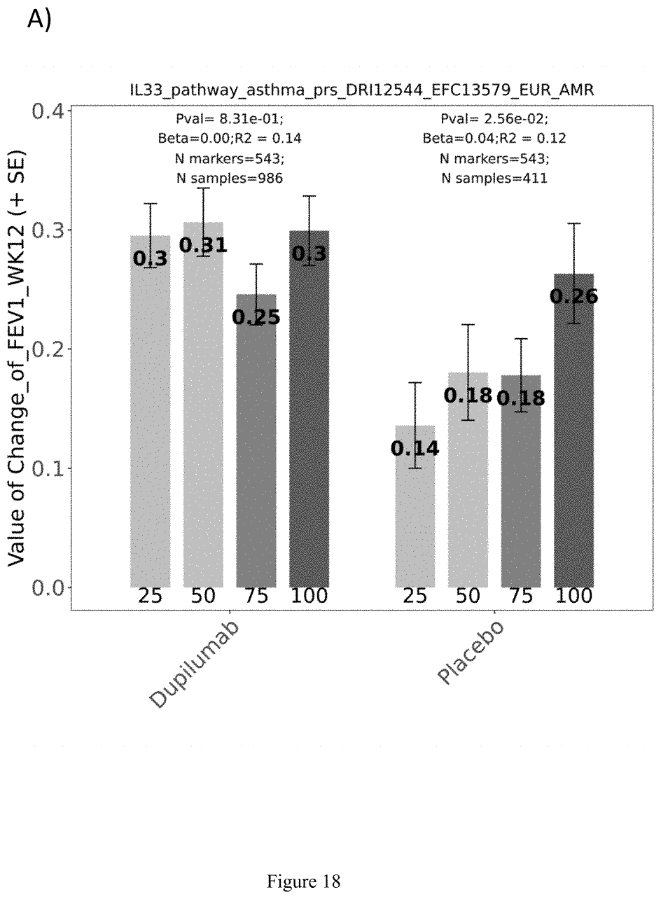

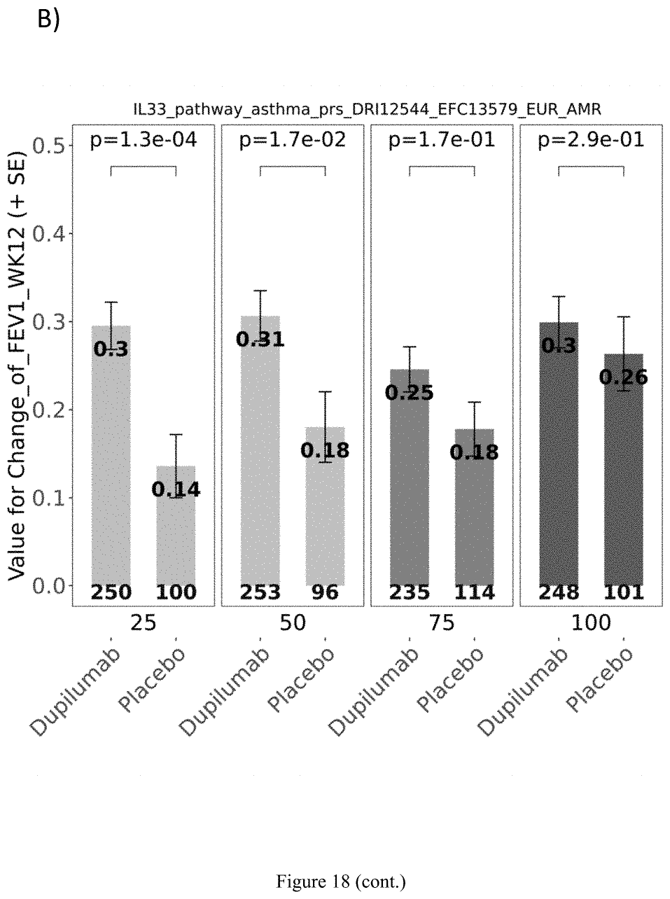

IL-33 Asthma PRS was significantly associated with an increased change of FEV1 at week 12 in placebo European and admixed American combined subjects in trials designated DR112544 and EFC13579. The IL-33 Asthma PRS against FEV1 change at week 12 for European and admixed American combined samples were regressed. Owing to increased sample size, a significant association between higher IL-33 Asthma PRSs and FEV1 increase was observed in the placebo arm, showing the high IL33 PRS asthma patients received greater benefits from LABAs or inhaled corticosteroid (graph in Panel A of FIG. 18). For the graph in Panel A, the IL-33 Asthma PRS divided into quartiles and ranked from lowest to the highest and the number inside each bar is the change in FEV1 at week 12. For the graph in Panel B, the IL-33 Asthma PRS divided into quartiles and ranked from lowest to the highest, the number at the bottom of each bar is the number of subjects in each group, and the ordinate is the change in FEV1 at week 12. The graph in Panel B illustrates a replication of this finding in European patients where patients with an IL-33 Asthma PRS in the 25th and 50th quartiles had significant dupilumab responses demonstrated by FEV1 changes. FIG. 18 shows that patients having an IL-33 Asthma PRS score in the bottom 25% in dupilumab arm have the most increased value of FEV1 compared to placebo arm.

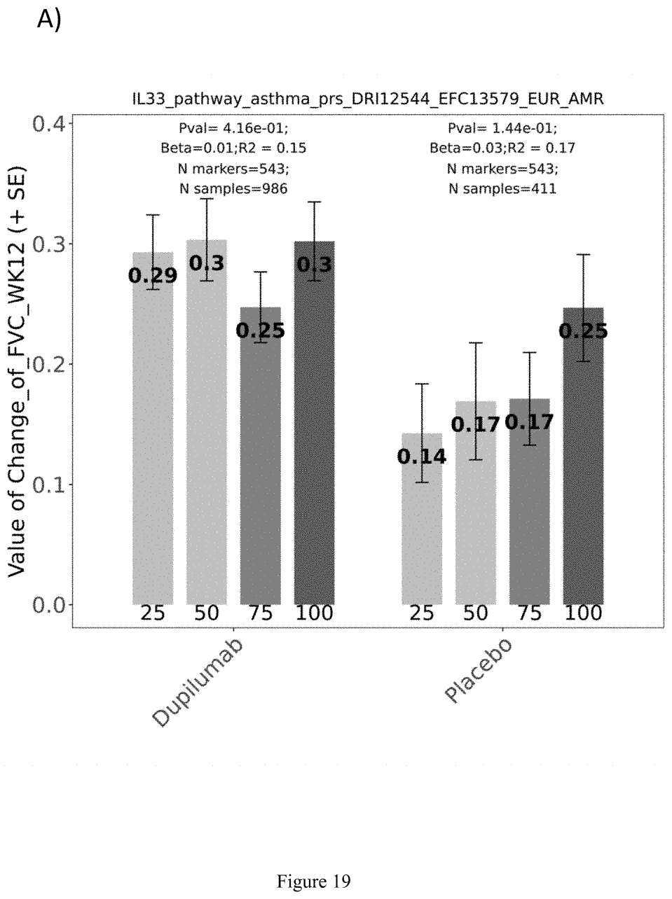

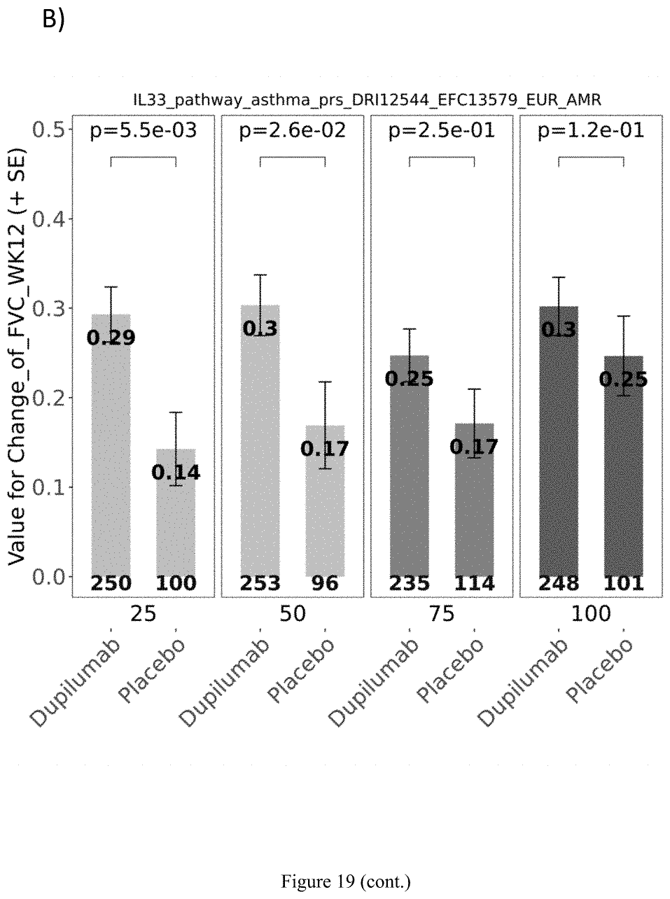

There was a relationship between IL-33 Asthma PRS and a change of FVC at week 12 in European and admixed American subjects. FIG. 19 shows a sample-wise distribution of variants. For the graph in Panel A, the IL-33 Asthma PRS divided into quartiles and ranked from lowest to the highest and the number inside each bar is the change in FEV1 at week 12. For the graph in Panel B, the IL-33 Asthma PRS divided into quartiles and ranked from lowest to the highest, the number at the bottom of each bar is the number of subjects in each group, and the ordinate is the change in FEV1 at week 12. Patients having IL-33 Asthma PRS score in the top 25% responded to dupilumab with less efficacy, compared to patients having lower IL-33 Asthma PRS scores and treated with dupilumab.

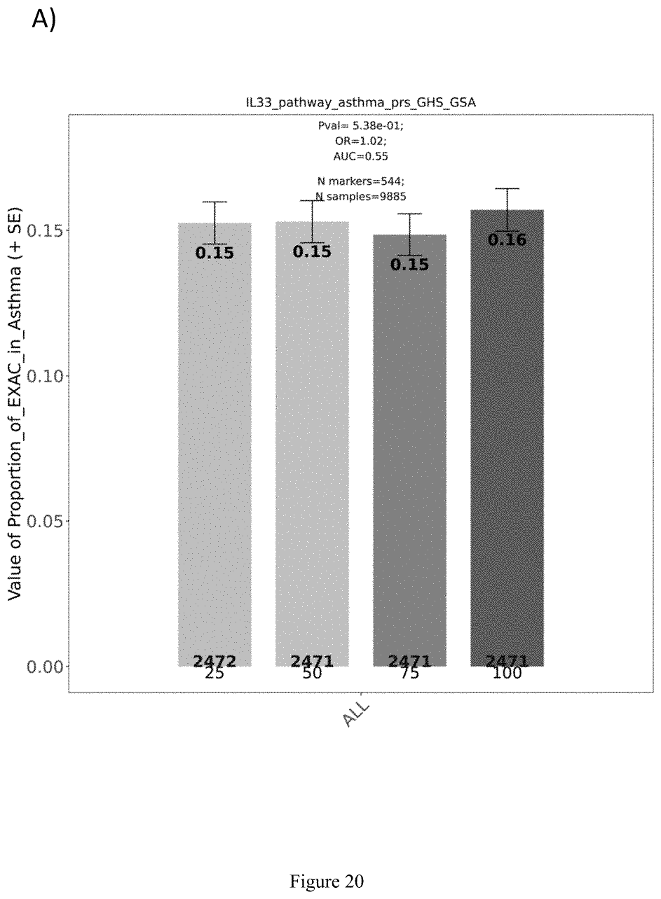

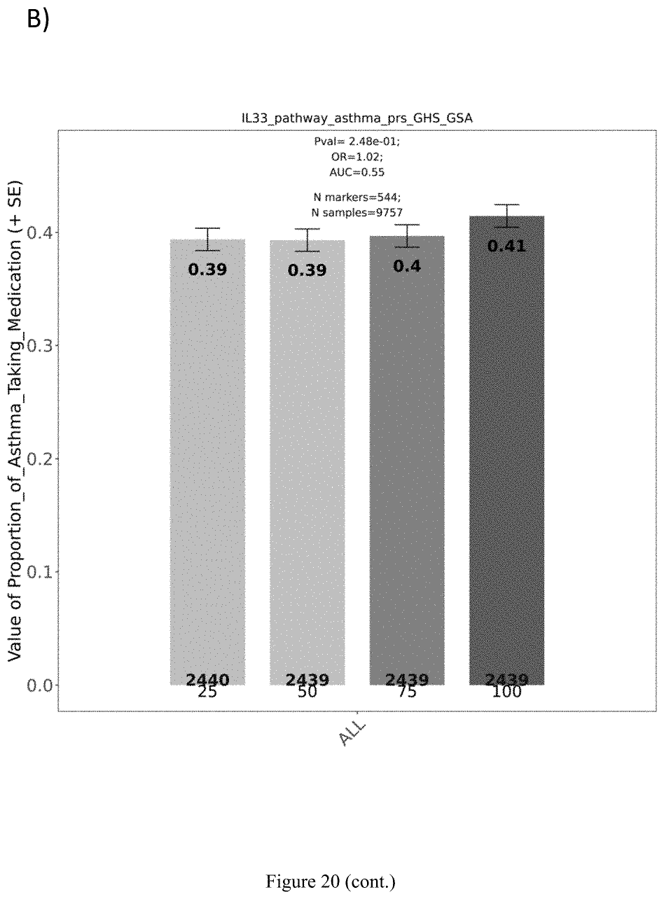

There was a relationship between IL-33 Asthma PRS and the proportion of subjects with asthma exacerbation in GHS_GSA European subjects. The data set forth in the graph in the Panel A of FIG. 20 demonstrate this finding. Panel A shows the lack of relationship between IL-33 Asthma PRS and the proportion of subjects with asthma exacerbation in GHS_GSA European subjects, and, Panel B shows the lack of a relationship between IL-33 Asthma PRS and the proportion of subjects with asthma taking medications. For the graph in Panel A, the IL-33 Asthma PRS divided into quartiles and ranked from lowest to the highest, the number at the bottom of each bar is the number of subjects in each group, and the number inside each bar is the proportion of subjects with asthma exacerbation. There is a relationship between an IL-33 Asthma PRS and the proportion of subjects with asthma taking medications. The data set forth in the graph in Panel B of FIG. 20 demonstrate this finding. For the graph in Panel B, the IL-33 Asthma PRS divided into quartiles and ranked from lowest to the highest, the number at the bottom of each bar is the number of subjects in each group, and the number inside each bar is the proportion of asthma subjects taking medication. FIG. 20 shows patients having IL-33 Asthma PRS score in the bottom 25% in dupilumab arm have the most increased value of FEV1 compared to the placebo arm.

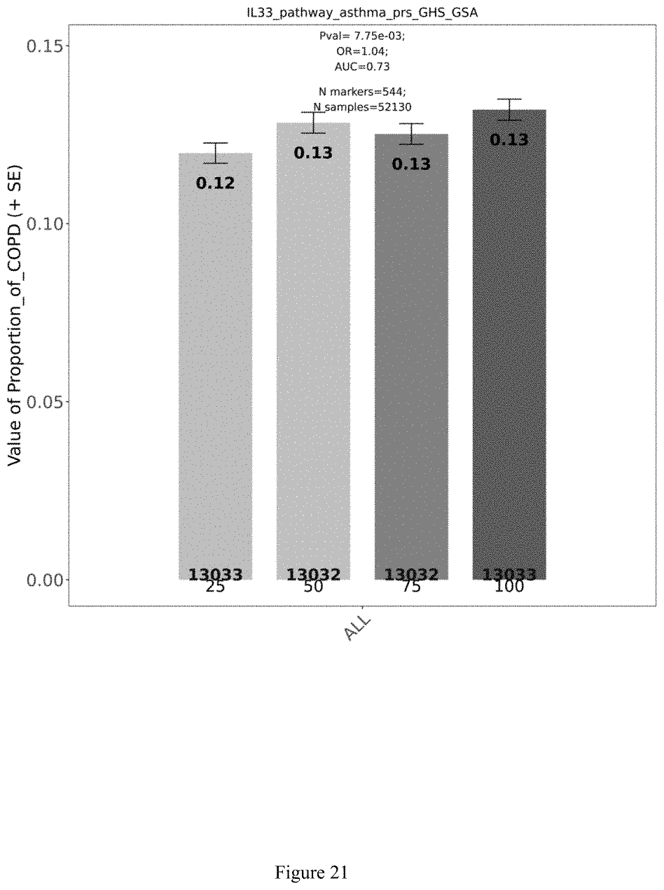

There was a relationship between an IL-33 Asthma PRS and the proportion of patients with chronic obstructive pulmonary disease (COPD) in GHS_GSA European patients. The graph set forth in FIG. 21 demonstrates this finding. The p value was 7.75e-03. For the graph, the IL-33 Asthma PRS divided into quartiles and ranked from lowest to the highest, the number at the bottom of each bar is the number of subjects in each group, and the number inside each bar is the proportion of subjects with COPD.

The PRS in this study was developed using GWAS data from individuals of European and admixed American ancestry. As GWAS data becomes available in more diverse populations, polygenic risk scores will likely improve over time for non-European populations as well.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A method of treating a subject having asthma or at risk of developing asthma, the method comprising:
administering an IL-33 antagonist to the subject when the subject's IL-33 asthma polygenic risk score (IL-33 Asthma PRS) is greater than or equal to a threshold IL-33 Asthma PRS, or
administering an interleukin-4 receptor alpha antagonist and/or an interleukin-13 receptor antagonist to the subject when the subject's IL-33 Asthma PRS is less than a threshold IL-33 Asthma PRS,
wherein the IL-33 Asthma PRS comprises a weighted aggregate of a plurality of genetic variants near or in the IL-33 and IL1RL1 genes associated with asthma.

2. A method of determining whether a subject should be administered an interleukin-4 receptor alpha antagonist, an interleukin-13 receptor antagonist, and/or an interleukin-33 (IL-33) antagonist for treatment of asthma, the method comprising:
determining or having determined the subject's IL-33 asthma polygenic risk score (IL-33 Asthma PRS), wherein the IL-33 Asthma PRS comprises a weighted aggregate of a plurality of genetic variants near or in the IL-33 and IL1RL1 genes associated with asthma; and
determining or having determined whether the subject's IL-33 Asthma PRS is greater than or equal to a threshold IL-33 Asthma PRS; and
when the subject's IL-33 Asthma PRS is greater than or equal to the threshold IL-33 Asthma PRS, the subject should be administered the IL-33 antagonist; or
when the subject's IL-33 Asthma PRS is less than the threshold IL-33 Asthma PRS, the subject should be administered the interleukin-4 receptor alpha antagonist and/or the interleukin-13 receptor antagonist.

3. A method of determining whether a subject having asthma should be administered a standard amount or greater of a composition for treating asthma exacerbation, comprising:
determining or having determined the subject's IL-33 asthma polygenic risk score (IL-33 Asthma PRS), wherein the IL-33 Asthma PRS comprises a weighted aggregate of a plurality of genetic variants near or in the IL-33 and IL1RL1 genes associated with asthma; and
when the subject's IL-33 Asthma PRS is greater than or equal to a threshold IL-33 Asthma PRS, the subject should be administered an interleukin-4 receptor alpha antagonist and/or an interleukin-13 receptor antagonist and a standard amount or greater of a composition for treating asthma exacerbation.

4. The method according to claim 1, wherein the interleukin-4 receptor alpha antagonist and/or the interleukin-13 receptor antagonist is dupilumab.

5. The method according to claim 1, wherein the IL-33 antagonist is itepekimab.

6. The method according to claim 1, wherein the threshold IL-33 Asthma PRS is the top 50% within a reference population.

7. The method according to claim 1, wherein the threshold IL-33 Asthma PRS is the top quintile within a reference population.

8. The method according to claim 1, wherein the threshold IL-33 Asthma PRS is the top decile within a reference population.

9. The method according to claim 1, wherein an endpoint of the IL-33 Asthma PRS is an increased annual exacerbation rate.

10. The method according to claim 1, wherein an endpoint of the IL-33 Asthma PRS is a loss of asthma control.

11. The method according to claim 1, wherein the reference population comprises at least 100 subjects.

12-14. (canceled)

15. The method according to claim 1, wherein the reference population is enriched for members of an ancestry group.

16. The method according to claim 15, wherein the ancestry group comprises a European ancestry group, an African ancestry group, an admixed American ancestry group, an East Asian ancestry group, or a South Asian ancestry group.

17-18. (canceled)

19. The method according to claim 1, wherein the plurality of genetic variants comprises a single nucleotide polymorphism (SNP), an insertion, a deletion, a structural variant, or a copy-number variation.

20. The method according to claim 1, wherein the plurality of genetic variants is determined by calculating a genetic variant performance in the reference population and selecting the highest performing genetic variants.

21. The method according to claim 20, wherein the genetic variant performance is calculated with respect to a strength of association and/or a probability distribution.

22. The method according to claim 1, wherein the IL-33 Asthma PRS is calculated using an LDpred method.

23. (canceled)

24. The method according to claim 1, wherein the IL-33 Asthma PRS is calculated using a pruning and thresholding method.

25-26. (canceled)

27. The method according to claim 1, wherein the plurality of genetic variants comprises at least 2 genetic variants.

28. (canceled)


SEQUENCE LISTING (10 sequences)

SEQ ID NO: 1 - protein, 124 aa, synthetic construct
  REGION 1..124: note = Dupilumab HCVR
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
  REGION 1..8: note = Dupilumab HCDR1
GFTFRDYA

SEQ ID NO: 4 - protein, 8 aa, synthetic construct
  REGION 1..8: note = Dupilumab HCDR2
ISGSGGNT

SEQ ID NO: 5 - protein, 18 aa, synthetic construct
  REGION 1..18: note = Dupilumab HCDR3
AKDRLSITIRPRYYGLDV

SEQ ID NO: 6 - protein, 11 aa, synthetic construct
  REGION 1..11: note = Dupilumab LCDR1
QSLLYSIGYNY

SEQ ID NO: 7 - skipped

SEQ ID NO: 8 - protein, 9 aa, synthetic construct
  REGION 1..9: note = Dupilumab LCDR3
MQALQTPYT

SEQ ID NO: 9 - protein, 451 aa, synthetic construct
  REGION 1..451: note = Dupilumab heavy chain
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, synthetic construct
  REGION 1..219: note = Dupilumab light chain
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC